(19) United States
(12) Patent Application Publication
(10) Pub. No.: US 2020/0337777 A1
(43) Pub. Date: Oct. 29, 2020

(54) SYSTEMS AND METHODS FOR SURGICAL ROUTE PLANNING

(71) Applicant: SHANGHAI UNITED IMAGING HEALTHCARE CO., LTD., Shanghai (CN)

(72) Inventors: Jian LIU, Shanghai (CN); Yun WANG, Shanghai (CN); Xiao FANG, Shanghai (CN); Liuzhu TONG, Shanghai (CN); Liangfan ZHU, Shanghai (CN); Gang CHEN, Shanghai (CN)

(73) Assignee: SHANGHAI UNITED IMAGING HEALTHCARE CO., LTD., Shanghai (CN)

(21) Appl. No.: 16/926,661

(22) Filed: Jul. 11, 2020

Related U.S. Application Data
(63) Continuation of application No. PCT/CN2019/071490, filed on Jan. 11, 2019.

(30) Foreign Application Priority Data
Jan. 11, 2018 (CN) 201810026525.0
May 29, 2018 (CN) 201810529406.7
May 31, 2018 (CN) 201810549359.2
Jun. 13, 2018 (CN) 201810609189.2

(51) Int. Cl. A61B 34/10; A61B 34/20; A61B 34/00; A61B 34/30; A61B 90/00; G16H 15/00; G16H 40/63; G16H 40/67

(52) U.S. Cl. CPC A61B 34/10; A61B 2090/3762; A61B 34/25; A61B 34/30; A61B 90/39; A61B 90/37; G16H 15/00; G16H 40/63; G16H 40/67; A61B 2034/107; A61B 2034/2068; A61B 2034/2055; A61B 2090/3937; A61B 2090/378; A61B 34/20

(57) ABSTRACT

A method for surgical route planning is provided. The method may include one or more of the following operations. A first image of a subject may be obtained. The first image may be generated based on first scan data acquired by a first imaging device in a first coordinate system. A first route in the first image may be determined. The first route may extend from a first point of the subject to a second point of the subject in the first coordinate system. The first route in the first coordinate system may be transformed to a second route in a second coordinate system related to maneuvering of a surgical equipment. An instruction to perform a surgical operation on the subject along the second route in the second coordinate system may be transmitted to the surgical equipment.

### CROSS-REFERENCE TO RELATED APPLICATIONS

This application is a continuation of International Patent Application No. PCT/CN2019/071490, filed on Jan. 11, 2019, which claims priority of Chinese Patent Application No. 201810609189.2 filed on Jun. 13, 2018, Chinese Patent Application No. 201810549359.2 filed on May 31, 2018, Chinese Patent Application No. 201810529406.7 filed on May 29, 2018, and Chinese Patent Application No. 201810026525.0 filed on Jan. 11, 2018, the entire contents of each of which are hereby incorporated by reference.

### TECHNICAL FIELD

The present disclosure generally relates to surgical route planning, and more particularly, relates to methods and systems for planning a surgical route for a surgical robot.

### BACKGROUND

Recently, automatic or semi-automatic surgical equipment, such as a surgical robot is increasingly used to perform a surgical operation on a patient. For example, the surgical robot may perform a puncture on the patient automatically based on a user instruction or a computer instruction. Normally, the automatic or semi-automatic surgical equipment may need to receive a planned route and perform the surgical operation along the route. The route may be planned based on a condition of the patient, which may need to be precise and suitable for the patient, otherwise the surgical operation may cause harm to the patient. Therefore, it is desirable to provide effective systems and methods for surgical route planning so as to guarantee the treatment effect.

### SUMMARY

In some aspects of the present disclosure, a system for surgical route planning is provided. The system may include at least one processor and at least one storage medium. The at least one storage medium may store a set of instructions for surgical route planning. When the at least one processor executes the set of instructions, the at least one processor may be directed to perform one or more of the following operations. The at least one processor may obtain a first image of a subject, the first image being generated based on first scan data acquired by a first imaging device in a first coordinate system. The at least one processor may determine a first route in the first image, the first route extending from a first point of the subject to a second point of the subject in the first coordinate system. The at least one processor may transform the first route in the first coordinate system to a second route in a second coordinate system related to maneuvering of a surgical equipment. And the at least one processor may transmit an instruction to the surgical equipment to perform a surgical operation on the subject along the second route in the second coordinate system.

In some embodiments, to determine the first route in the first image, the at least one processor is further configured to direct the system to perform additional operations including: identifying a lesion of the subject based on the first image; determining an operation area on a body surface of the subject and the second point based on the lesion; and determining the first route based on the operation area and the second point, wherein the first point is within the operation area.

In some embodiments, to determine the first route based on the operation area and the second point, the at least one processor is further configured to direct the system to perform additional operations including: determining a plurality of candidate routes based on the operation area and the second point, each of the plurality of candidate routes extending from a point within the operation area to the second point; and selecting the first route from the plurality of candidate routes.

In some embodiments, the selection of the first route is based on one or more selection criteria. The one or more selection criteria are related to at least one of lengths of the plurality of candidate routes, directions of the plurality of candidate routes, or whether the plurality of candidate routes pass through one or more critical tissues of the subject.

In some embodiments, to determine the first route in the first image, the at least one processor is further configured to direct the system to perform additional operations including: identifying a lesion of the subject based on the first image; obtaining a plurality of historical treatment records of a plurality of sample subjects, each of the plurality of historical treatment records including a historical route with respect to a historical lesion of one of the plurality of sample subjects; and determining the first route based on the lesion and the plurality of historical treatment records.

In some embodiments, to determine the first route based on the lesion and the plurality of historical records, the at least one processor is further configured to direct the system to perform additional operations including: determining a similarity degree between the lesion and each of the plurality of historical lesions; and determining the first route based on the similarity degrees.

In some embodiments, to determine the first route in the first image, the at least one processor is further configured to direct the system to perform additional operations including: receiving one or more operation parameters related to the first route from a user; and determining the first route based at least one of the one or more operation parameters.

In some embodiments, to transform the first route in the first coordinate system to the second route in the second coordinate system related to maneuvering of the surgical equipment, the at least one processor is further configured to direct the system to perform additional operations including: determining a first transformation relationship between the first coordinate system and a reference coordinate system; determining a second transformation relationship between the second coordinate system and the reference coordinate system; determining a third transformation relationship between the first coordinate system and the second coordinate system based on the first transformation relationship and the second transformation relationship; and transforming the first route in the first coordinate system to the second route in the second coordinate system related to maneuvering of a surgical equipment based on the third transformation relationship.

In some embodiments, to determine the first transformation relationship between the first coordinate system and the reference coordinate system, the at least one processor is further configured to direct the system to perform additional operations including: determining a plurality of first coordinates of a plurality of markers placed on a body surface of the subject in the first coordinate system; determining a plurality of reference coordinates of the plurality of markers in the reference coordinate system; and determining the first transformation relationship between the first coordinate system and the reference coordinate system based on plurality of first coordinates and the plurality of reference coordinates.

In some embodiments, to determine the second transformation relationship between the second coordinate system and the reference coordinate system, the at least one processor is further configured to direct the system to perform additional operations including: determining one or more second coordinates of the one or more markers in the second coordinate system; and determining the second transformation relationship between the second coordinate system and the reference coordinate system based on the one or more second coordinates and the one or more reference coordinates.

In some embodiments, the at least one processor is further configured to direct the system to perform additional operations including: determining a first relative position of the surgical equipment with respect to a first position at which the subject is located when the first scan data is acquired; determining a second relative position of the surgical equipment with respect to a second position at which the subject is located during the surgical operation; and upon detecting that a difference between the first relative position and the second relative position exceeds a predetermined threshold, transmitting an instruction to the surgical equipment to move to a target position, the target position having a substantially same relative position with respect to the second position of the subject as the first relative position with respect to the first position.

In some embodiments, at least one of the first relative position or the second relative position is determined by tracking positions of at least one of one or more first makers placed on a body surface of the subject or one or more second markers placed on the surgical equipment.

In some embodiments, the at least one processor is further configured to direct the system to perform additional operations including: obtaining a second image of the subject after the surgical operation, the second image being generated based on second scan data acquired by the first imaging device; and determining an operation result based on the second image.

In some embodiments, to obtain the second image of the subject after the surgical equipment, the at least one processor is further configured to direct the system to perform additional operations including: transmitting an instruction to the first imaging device to move the subject into a detection tunnel of the first imaging device; determining a movement of the subject during moving the subject into the detection tunnel; and transmitting an instruction to the surgical equipment to move in a manner consistent with the movement of the subject.

In some embodiments, the at least one processor is further configured to direct the system to perform additional operations including: obtaining a third image of the subject, the third image being generated according to scan data acquired by a second imaging device during the surgical operation, the third image indicating a moving trajectory of the surgical equipment during the surgical operation; determining whether the moving trajectory of the surgical equipment deviates from the second route; and in response to a determination that the surgical equipment deviates from the second route, transmitting an instruction to the surgical equipment to terminate the surgical operation or adjust the surgical operation.

In some embodiments, the surgical equipment may be mounted on a first robotic arm of a surgical robot, and the second imaging device may be an ultrasonic imaging device mounted on a second robotic arm of the surgical robot.

In some embodiments, the surgical operation includes at least one of a puncture, a biopsy, an ablation, a grinding, a drilling, an implantation, or a suction.

In some aspects of the present disclosure, a method for surgical route planning is provided. The method may be implemented on a computing device having one or more processors and one or more storage media. The method may include one or more of the following operations. A first image of a subject may be obtained, the first image being generated based on first scan data acquired by a first imaging device in a first coordinate system. A first route in the first image may be determined, the first route extending from a first point of the subject to a second point of the subject in the first coordinate system. The first route in the first coordinate system may be transformed to a second route in a second coordinate system related to maneuvering of a surgical equipment. An instruction to perform a surgical operation on the subject along the second route in the second coordinate system may be transmitted to the surgical equipment.

In some aspects of the present disclosure, a non-transitory computer readable medium is provided. The non-transitory computer readable medium may include a set of instructions for surgical route planning. When at least one processor executes the set of instructions, the at least one processor may be directed to perform one or more of the following operations. The at least one processor may obtain a first image of a subject, the first image being generated based on first scan data acquired by a first imaging device in a first coordinate system. The at least one processor may determine a first route in the first image, the first route extending from a first point of the subject to a second point of the subject in the first coordinate system. The at least one processor may transform the first route in the first coordinate system to a second route in a second coordinate system related to maneuvering of a surgical equipment. And the at least one processor may transmit an instruction to the surgical equipment to perform a surgical operation on the subject along the second route in the second coordinate system.

In some aspects of the present disclosure, a system for surgical route planning is provided. The system may include an obtaining module, a determination module, a transformation module, and a transmission module. The obtaining module may be configured to obtain a first image of a subject, the first image being generated based on first scan data acquired by a first imaging device in a first coordinate system. The determination module may be configured to determine a first route in the first image, the first route extending from a first point of the subject to a second point of the subject in the first coordinate system. The transformation module may be configured to transform the first route in the first coordinate system to a second route in a second coordinate system related to maneuvering of a surgical equipment. The transmission module may be configured to transmit an instruction to the surgical equipment to perform a surgical operation on the subject along the second route in the second coordinate system.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary surgery system according to some embodiments of the present disclosure;

FIG. 2 is a schematic diagram illustrating exemplary hardware and/or software components of an exemplary computing device according to some embodiments of the present disclosure;

FIG. 3 is a schematic diagram illustrating exemplary hardware and/or software components of an exemplary mobile device according to some embodiments of the present disclosure;

FIG. 4 is a block diagram illustrating an exemplary processing device according to some embodiments of the present disclosure;

FIG. 5 is a flowchart illustrating an exemplary process for planning a surgical route for a surgical equipment according to some embodiments of the present disclosure;

FIG. 6 is a flowchart illustrating an exemplary process for determining a first route in a first image according to some embodiments of the present disclosure;

FIG. 7 is a flowchart illustrating another exemplary process for determining a first route in a first image according to some embodiments of the present disclosure;

FIG. 8 is a flowchart illustrating another exemplary process for transforming a first route in a first coordinate system to a second route in a second coordinate system according to some embodiments of the present disclosure;

FIG. 9 is a flowchart illustrating another exemplary process for monitoring a relative position of a surgical equipment with respect to a subject according to some embodiments of the present disclosure;

FIG. 10 is a flowchart illustrating another exemplary process for monitoring a moving trajectory of a surgical equipment during a surgical operation according to some embodiments of the present disclosure;

FIGS. 11A and 11B are schematic diagrams illustrating an exemplary surgical operation system according to some embodiments of the present disclosure; and

FIG. 12 is a schematic diagram illustrating an exemplary surgery system according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, section or assembly of different level in ascending order. However, the terms may be displaced by other expression if they achieve the same purpose.

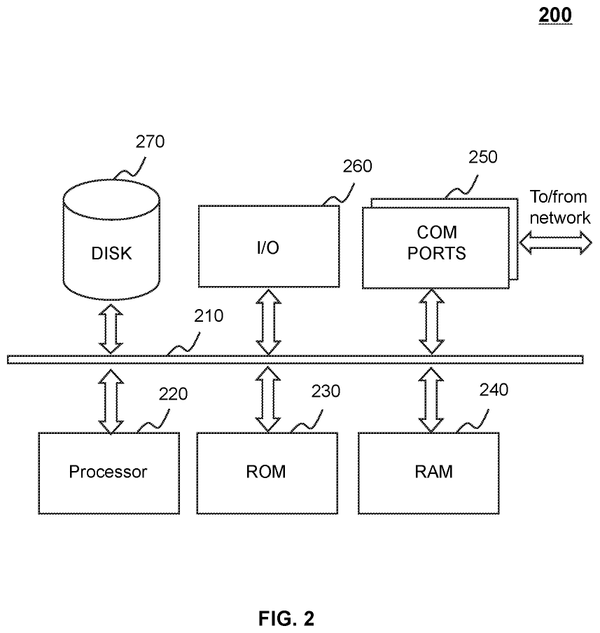

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., the processor 220 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The following description is provided to help better understanding the processing methods and/or systems. This is not intended to limit the scope the present disclosure. For persons having ordinary skills in the art, a certain amount of variations, changes, and/or modifications may be deducted under the guidance of the present disclosure. Those variations, changes, and/or modifications do not depart from the scope of the present disclosure.

Provided herein are systems and methods for planning a surgical route in surgeries, such as for disease diagnosis, disease treatment, or research purposes. The systems may perform the methods to obtain a first image of a subject. The first image may be generated based on first scan data acquired by a first imaging device in a first coordinate system. The systems may perform the methods to determine a first route in the first image, which may be a virtual planned surgical route in the first image corresponding to the surgical route. The systems and methods may transform the first route to a second route (i.e., the actual surgical route) in a second coordinate system related to maneuvering of a surgical equipment, and transmit an instruction to the surgical equipment to perform a surgical operation on the subject along the second route. In some embodiments, the systems may further perform the methods to monitor the relative position between the subject and the surgical equipment after the first scan data is acquired, monitor a moving trajectory of the surgical equipment during the surgical operation, and/or evaluate an operation result after the surgical operation. The systems and methods provided herein may ensure that the planned surgical route is precise and suitable for the subject and that the surgical operation is performed according to the planned surgical route, thus guaranteeing the treatment effect on the subject.

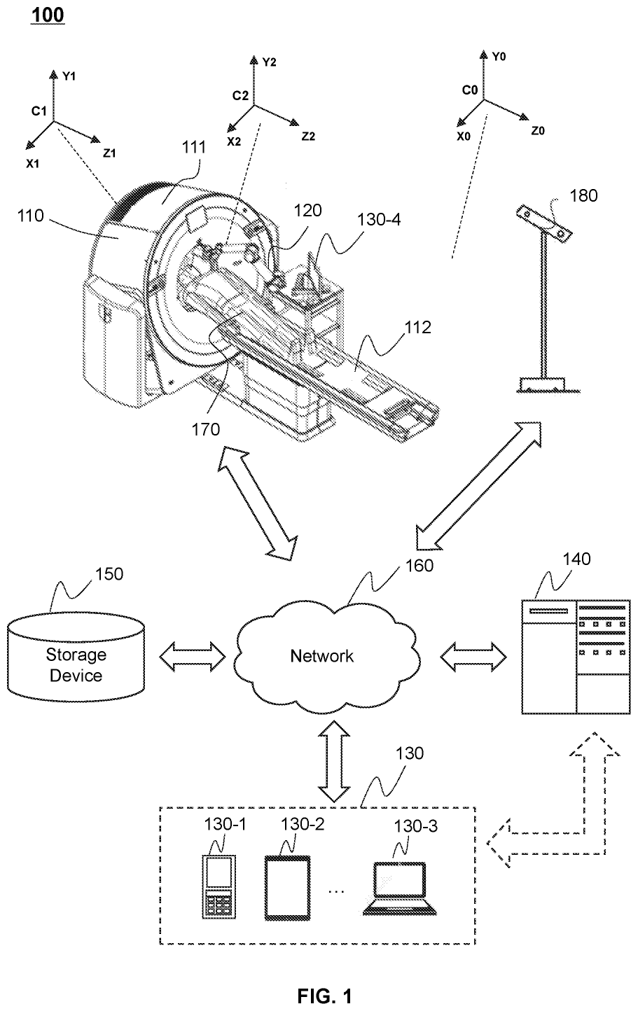

FIG. 1 is a schematic diagram illustrating an exemplary surgery system according to some embodiments of the present disclosure. The surgery system 100 may be configured to perform a surgical operation on a subject 170. Exemplary surgical operations may include a puncture, a biopsy, an ablation (e.g., a radiofrequency ablation), a grinding (e.g., a bone grinding), a drilling (e.g., a bone drilling), an implantation (e.g., a radioactive seed implantation), a suction, or the like. The subject 170 may include a user (e.g., a patient), a portion of the user (e.g., an organ and/or a tissue of the user), a man-made object (e.g., a phantom), etc.

As shown in FIG. 1, the surgery system 100 may include an imaging device 110, a surgical equipment 120, one or more terminals 130, a processing device 140, a storage device 150, a network 160, a subject 170, and a tracking device 180. The connection between the components in the surgery system 100 may be variable. Merely by way of example, as illustrated in FIG. 1, the imaging device 110 and/or the surgical equipment 120 may be connected to the processing device 140 through the network 160. As another example, the imaging device 110 may be connected to the processing device 140 directly. As a further example, the storage device 150 may be connected to the processing device 140 directly or through the network 160. As still a further example, the terminal 130 may be connected to the processing device 140 directly (as indicated by the bi-directional arrow in dotted lines linking the terminal 130 and the processing device 140) or through the network 160.

The imaging device 110 may be configured to perform a scan on the subject 170 to acquire scan data related to the subject 170 before, during, and/or after the surgical operation. In some embodiments, one or more images of the subject 170 may be reconstructed based on the scan data by the processing device 140. The image(s) may be used in, for example, planning the surgical operation, implementing the surgical operation, and/or evaluating of a result of the surgical operation. For example, the imaging device 110 may perform a scan on the subject 170 before the surgical operation and an image of the subject 170 may be generated based on the scan. The image may indicate a lesion of the subject 170 and be used as a basis for planning a surgical route of the surgical equipment 120. As another example, the imaging device 110 may scan the subject 170 during the surgical operation in real-time or periodically to monitor a moving trajectory of the surgical equipment 120.

The imaging device 110 may include a digital subtraction angiography (DSA) device, a magnetic resonance imaging (MRI) device, a computed tomography angiography (CTA) device, a positron emission tomography (PET) device, a single photon emission computed tomography (SPECT) device, a computed tomography (CT) device (e.g., a cone beam CT), a digital radiography (DR) device, or the like. In some embodiments, the imaging device 110 may be a multi-modality imaging device including, for example, a PET-CT device, a PET-MRI device, a SPECT-PET device, a DSA-MRI device, or the like.

In some embodiments, as illustrated in FIG. 1, the imaging device 110 may include a gantry 111, a table 112, a detecting tunnel (not shown), a radiation source (not shown), and a detector (not shown). The gantry 111 may support the detector and the radiation source. A subject may be placed on the table 112 for scanning. The radiation source may emit radioactive rays to the subject, and the detector may detect radiation rays (e.g., X-rays) emitted from the detecting tunnel. In some embodiments, the detector may include one or more detector units. The detector units may include a scintillation detector (e.g., a cesium iodide detector), a gas detector, etc. The detector unit may be include a single-row detector and/or a multi-rows detector.

The surgical equipment 120 may be configured to perform the surgical operation on the subject 170 automatically or semi-automatically. As used herein, an automatic surgical operation may refer to a surgical operation automatically performed by the surgical equipment 120. A semi-automatic surgical operation may refer to a surgical operation performed by the surgical equipment 120 with a user intervention. The user intervention may include, for example, providing information regarding the subject 170 (e.g., a location of a lesion of the subject 170), providing information regarding the surgical operation (e.g., a parameter related to the surgical operation), or the like, or a combination thereof. In some embodiments, the surgical equipment 120 may refer to an actuating mechanism that actually performs the surgical operation on the subject. For example, the surgical equipment 120 may include a biopsy needle, a puncture needle, an ablation needle, an ablation probe, a drill bit, or the like, or any combination thereof. Alternatively, the surgical equipment 120 may refer to the actuating mechanism and an equipment that assembled with the actuating mechanism. For example, the surgical equipment 120 may include a robotic arm or a surgical robotic assembled with the actuating mechanism (e.g., a puncture needle).

In some embodiments, the surgical equipment 120 may be a puncture device. The puncture device may include a base, a puncture unit, a movement control mechanism, and/or a position-limiting mechanism. The puncture unit may be configured to perform a puncture on the subject 170. The base may be configured to support one or more components of the puncture device. The movement control mechanism may be assembled on the base and configured to control a movement of the puncture unit. The position-limiting mechanism may be movably mounted on the base and configured to limit a position of the movement control mechanism during a movement of the movement control mechanism. Optionally, the puncture device may further include one or more other components, such as a firing actuator, a guiding device, a location detection device, a positioning mechanism, and a mounting mechanism.

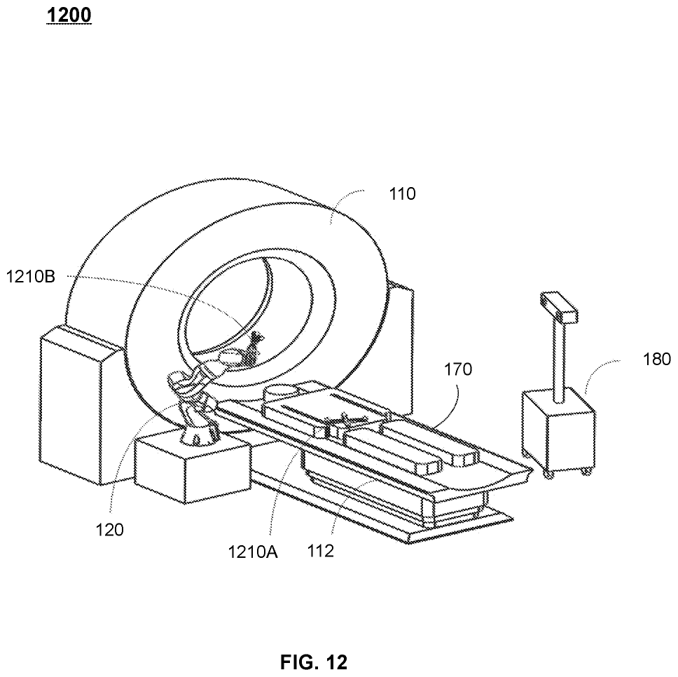

The tracking device 180 may be configured to track the positions of one or more components of the surgery system 100 (e.g., the imaging device 110, the surgical equipment 120, and/or the subject 170) and/or determine relative positions between two or more components of the surgery system 100. In some embodiments, the tracking device 180 may be an image acquisition device that captures an image or a video of the one or more components of the surgery system 100. For example, the tracking device 180 may be a camera (e.g., a binocular camera or a video camera), a mobile phone assembled with the camera, or the like, or any combination thereof. The image or video captured by the tracking device 180 may indicate the positions of the one or more components in the surgery system 100 as well as a relative position between two or more of the components. In some embodiments, the tracking device 180 may determine the position of the one or more components by tracking one or more markers placed on the one or more components. Details regarding the tracking device 180 may be found elsewhere in the present disclosure (e.g., FIG. 12 and the relevant descriptions thereof).

In some embodiments, as illustrated in FIG. 1, the imaging device 110, the surgical equipment 120, and the surgery system 100 may correspond to a coordinate system C1 (also referred to as a first coordinate system), a coordinate system C2 (also referred to as a second coordinate system), and a coordinate system C0 (also referred to as a reference coordinate system), respectively. The coordinate systems C0, C1, and C2 may have any number of dimensions and the dimension(s) may be in any direction. The origins of the coordinate systems C0, C1, and C2 may be located at any suitable position.

Merely by way of example, the coordinate systems C0, C1, and C2 are both be a Cartesian coordinate system including three dimensions as shown in FIG. 1. In some embodiments, the origin of the coordinate system C1 may be located at the center of the gantry 111 of the imaging device 110. The coordinate system C1 may include a Z1-axis, an X1-axis, and an Y1-axis, wherein the Z1-axis is parallel with the moving direction of the table 112, and the X1-axis and the Y1-axis forms a plane perpendicular to the Z1-axis. The origin of the coordinate system C2 may be located at any point on the surgical equipment 120. The coordinate system C2 may include a Z2-axis, an X2-axis, and an Y2-axis, which are parallel with the Z1-axis, the X1-axis, and the Y1-axis, respectively. The origin of the coordinate system C0 may be located at any point in the surgery system 100, for example, a point on the tracking device 180. The coordinate system C0 may include a Z0-axis, an X0-axis, and an Y0-axis, which are parallel with the Z1-axis, the X1-axis, and the Y1-axis, respectively.

The terminal 130 may be configured to realize an interaction between a user and one or more components of the surgery system 100. For example, the terminal 130 may have a user interface (UI) for the user to input an instruction to the surgical equipment 120 to perform a surgical operation on the subject 170. As another example, the terminal 130 may display one or more images acquired by the surgery system 100 to the user. The terminal 130 may include a mobile device 130-1, a tablet computer 130-2, a laptop computer 130-3, a display 130-4, or the like, or any combination thereof. In some embodiments, the mobile device 130-1 may include a smart home device, a wearable device, a mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, a footgear, eyeglasses, a helmet, a watch, clothing, a backpack, a smart accessory, or the like, or any combination thereof. In some embodiments, the mobile device may include a mobile phone, a personal digital assistance (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™, etc. In some embodiments, the terminal 130 may be part of the processing device 140.

The processing device 140 may process data and/or information related to the surgery system 100, for example, information obtained from the imaging device 110, the surgical equipment 120, the terminal 130, the storage device 150, and/or the tracking device 180. For example, the processing device 140 may receive scan data of the subject 170 from the imaging device 110 and reconstruct an image of the subject 170 based on the scan data. As another example, the processing device 140 may further determine a surgical route for the surgical equipment 120 based on the reconstructed image of the subject 170. In some embodiments, the processing device 140 may be a single server or a server group. The server group may be centralized or distributed. In some embodiments, the processing device 140 may be local or remote. For example, the processing device 140 may access information and/or data stored in the imaging device 110, the surgical equipment 120, the terminal 130, and/or the storage device 150 via the network 160. As another example, the processing device 140 may be directly connected to the imaging device 110, the terminal 130 and/or the storage device 150 to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented by a computing device 200 having one or more components as illustrated in FIG. 2.

The storage device 150 may store data, instructions, and/or any other information. In some embodiments, the storage device 150 may store data obtained from the imaging device 110, the surgical equipment 120, the terminal 130, and the processing device 140. In some embodiments, the storage device 150 may store data and/or instructions that the processing device 140 and/or the terminal 130 may execute or use to perform exemplary methods described in the present disclosure. In some embodiments, the storage device 150 may include a mass storage, a removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 160 to communicate with one or more other components in the surgery system 100 (e.g., the processing device 140, the terminal 130, etc.). One or more components in the surgery system 100 may access the data or instructions stored in the storage device 150 via the network 160. In some embodiments, the storage device 150 may be directly connected to or communicate with one or more other components in the surgery system 100 (e.g., the imaging device 110, the processing device 140, the terminal 130, etc.). In some embodiments, the storage device 150 may be part of the processing device 140.

The network 160 may include any suitable network that can facilitate exchange of information and/or data in the surgery system 100. In some embodiments, one or more components of the surgery system 100 (e.g., the imaging device 110, the surgical equipment 120, the terminal 130, the processing device 140, the storage device 150, and/or the tracking device 180) may communicate with each other via the network 160. For example, the processing device 140 may obtain historical treatment records from the storage device 150 via the network 160. As another example, the imaging device 110 and/or the surgical equipment 120 may obtain user instructions from the terminal 130 via the network 160. The network 160 may include a public network (e.g., the Internet), a private network (e.g., a local area network (LAN), a wide area network (WAN), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, witches, server computers, and/or any combination thereof. Merely by way of example, the network 160 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 160 may include one or more network access points. For example, the network 160 may include wired and/or wireless network access points such as base stations and/or internet exchange points through which one or more components of the surgery system 100 may be connected to the network 160 to exchange data and/or information.

It should be noted that the above description of the surgery system 100 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the surgery system 100 may include one or more additional components. Additionally or alternatively, one or more components of the surgery system 100 described above may be omitted. For example, the tracking device 180 may be omitted. As another example, the surgery system 100 may further include a second imaging device other than the imaging device 110, which is configured to capture an image of the subject during the surgical operation. In some embodiments, the surgery system 100 may further include a distance measuring device configured to measure a distance from the distance measuring device to one or more components of the surgery system 100. Merely by way of example, the distance measuring device may measure a distances from the surgical equipment 120 and the subject 170 to the distance measuring device, wherein the distances may be used for determining the positions of the surgical equipment 120 and the subject 170. Optionally, the distance measuring device may be integrated into the tracking device 180.

FIG. 2 is a schematic diagram illustrating exemplary hardware and/or software components of an exemplary computing device according to some embodiments of the present disclosure. In some embodiments, one or more components of the surgery system 100 may be implemented on one or more components of the computing device 200. Merely by way of example, the processing device 140 and/or the terminal 130 may be implemented one or more components of the computing device 200, respectively.

As illustrated in FIG. 2, the computing device 200 may include a communication bus 210, a processor 220, a storage, an input/output (I/O) 260, and a communication port 250. The processor 220 may execute computer instructions (e.g., program code) and perform functions of one or more components of the surgery system 100 (e.g., the processing device 140) in accordance with techniques described herein. The computer instructions may include, for example, routines, programs, objects, components, data structures, procedures, modules, and functions, which perform particular functions described herein. In some embodiments, the processor 220 may include interface circuits and processing circuits therein. The interface circuits may be configured to receive electronic signals from the communication bus 210, wherein the electronic signals encode structured data and/or instructions for the processing circuits to process. The processing circuits may conduct logic calculations, and then determine a conclusion, a result, and/or an instruction encoded as electronic signals. Then the interface circuits may send out the electronic signals from the processing circuits via the communication bus 210.

Merely for illustration, only one processor 220 is described in the computing device 200. However, it should be noted that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both step A and step B, it should be understood that step A and step B may also be performed by two or more different processors jointly or separately in the computing device 200 (e.g., a first processor executes step A and a second processor executes step B, or the first and second processors jointly execute steps A and B).

The storage may store data/information related to the surgery system 100, such as information obtained from the imaging device 110, the surgical equipment 120, the terminal 130, the storage device 150, the tracking device 180, and/or any other component of the surgery system 100. In some embodiments, the storage may include a mass storage, a removable storage, a volatile read-and-write memory, a random access memory (RAM) 240, a read-only memory (ROM) 230, a disk 270, or the like, or any combination thereof. In some embodiments, the storage may store one or more programs and/or instructions to perform exemplary methods described in the present disclosure. For example, the storage may store a program for the processing device 140 for operating a surgery.

The I/O 260 may input and/or output signals, data, information, etc. In some embodiments, the I/O 260 may enable a user interaction with the computing device 200. In some embodiments, the I/O 260 may include an input device and an output device. Examples of the input device may include a keyboard, a mouse, a touch screen, a microphone, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touch screen, or the like, or a combination thereof.

The communication port 250 may be connected to a network (e.g., the network 160) to facilitate data communications. The communication port 250 may establish connections between the computing device 200 (e.g., the processing device 140) and the imaging device 110, the surgical equipment 120, the terminal 130, and/or the storage device 150. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G, etc.), or the like, or a combination thereof. In some embodiments, the communication port 250 may be and/or include a standardized communication port, such as RS232, RS485, etc. In some embodiments, the communication port 250 may be a specially designed communication port. For example, the communication port 250 may be designed in accordance with the digital imaging and communications in medicine (DICOM) protocol.

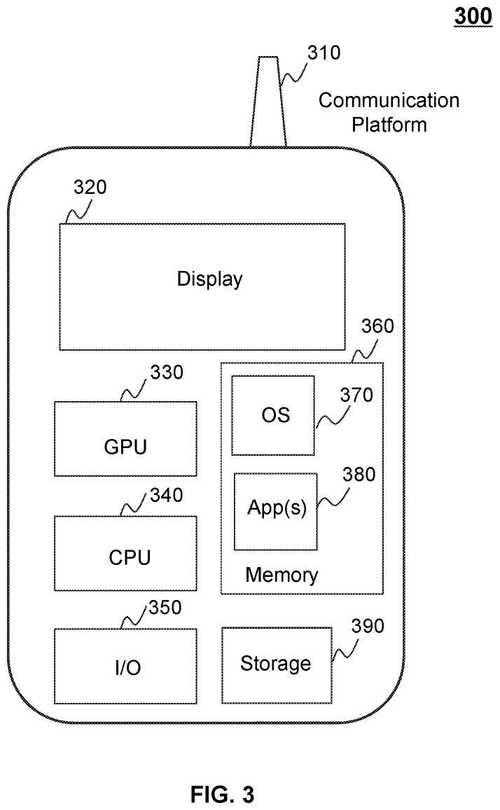

FIG. 3 is a schematic diagram illustrating exemplary hardware and/or software components of an exemplary mobile device according to some embodiments of the present disclosure. In some embodiments, one or more components of the surgery system 100 may be implemented on one or more components of the mobile device 300. Merely by way of example, the terminal 130 may be implemented on one or more components of the mobile device 300.

As illustrated in FIG. 3, the mobile device 300 may include a communication platform 310, a display 320, a graphic processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS™, Android™ Windows Phone™, etc.) and one or more applications 380 may be loaded into the memory 360 from the storage 390 in order to be executed by the CPU 340. The applications 380 may include a browser or any other suitable mobile apps for receiving and rendering information relating to the surgery system 100. User interactions with the information stream may be achieved via the I/O 350 and provided to one or more components of the surgery system 100 via the network 160.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

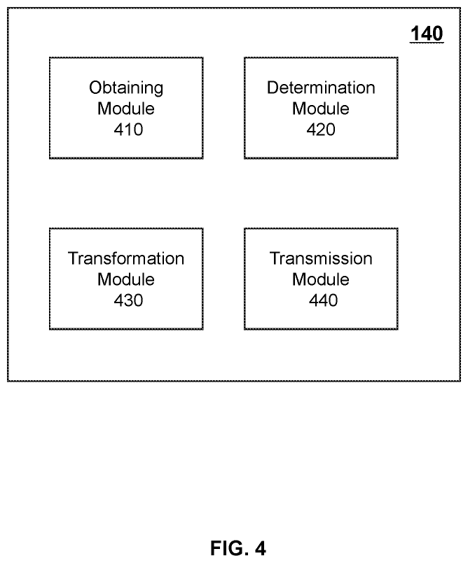

FIG. 4 is a block diagram illustrating an exemplary processing device according to some embodiments of the present disclosure. The processing device 140 may include an obtaining module 410, a determination module 420, a transformation module 430, and a transmission module 440. One or more of the modules of the processing device 140 may be interconnected. The connection(s) may be wireless or wired.

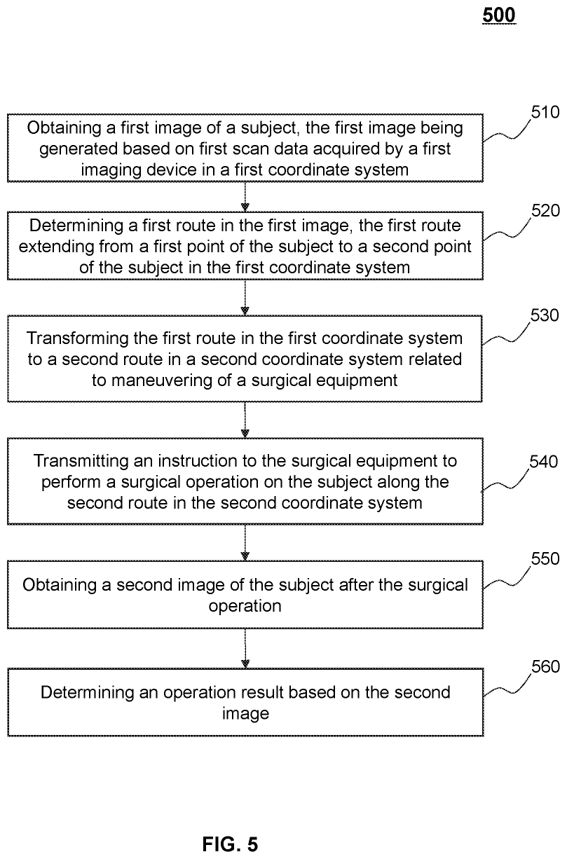

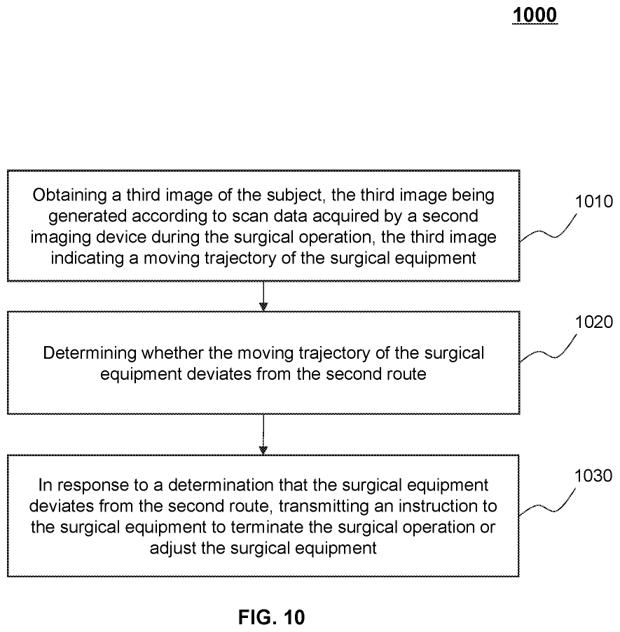

The obtaining module 410 may be configured to obtain information related to the surgery system 100. For example, the obtaining module 410 may obtain one or more images of a subject. The image(s) may include a first image, a second image, and/or a third image of the subject. The first image may be generated based on first scan data acquired by a first imaging device (e.g., the imaging device 110) in a first coordinate system before the surgical equipment 120 performs a surgical operation on the subject. The second image may be generated based on second scan data acquired by the first imaging device after the surgical operation. The third image may be captured by a second imaging device (e.g., an ultrasonic imaging device) during the surgical operation. Details regarding the obtaining of the first image, the second image, and/or the third image may be found elsewhere in the present disclosure (e.g., FIGS. 5 and 10 and the relevant descriptions thereof).

The determination module 420 may be configured to determine a first route in the first image. The first route may refer a virtual planned surgical route in the first image in the first coordinate system that corresponds to a surgical route of the surgical equipment 120. In some embodiments, the determination module 420 may determine a lesion of the subject based on the first image, and further determine the first route based on the lesion. For example, the determination module 420 may determine the first route by comparing the lesion and a plurality of historical lesions in a plurality of historical treatment records. In some embodiments, the determine the first route under a user intervention, for example, based on one or more parameters related to the first route inputted by a user of the surgery system 100. Details regarding the determination of the first route may be found elsewhere in the present disclosure (e.g., FIG. 520 and the relevant descriptions thereof).

In some embodiments, the determination module 420 may be configured to determine an operation result based on the second image. The operation result may include, for example, whether the lesion of the subject is removed by the surgical operation, whether a proportion of the lesion is removed by the surgical operation, whether the surgical equipment reaches an end point of the surgical route, or the like, or any combination thereof. In some embodiments, determination module 420 may determine the operation result by comparing the first image (or the first scan data) with the second image (or the second scan data). Details regarding the determination of the operation result may be found elsewhere in the present disclosure (e.g., operation 560 and the relevant descriptions thereof).

The transformation module 430 may be configured to transform the first route in the first coordinate system to a second route in a second coordinate system related to maneuvering of the surgical equipment. As used herein, the second route may refer the actual planned surgical route of the surgical equipment 120 in the second coordinate system. The surgical equipment may be maneuvered along the second route during the surgical operation. In some embodiments, the transformation module 430 may transform the first route to the second route based on a transformation relationship between the first coordinate system and the second coordinate system. Details regarding the transformation of the first route to the second route may be found elsewhere in the present disclosure (e.g., operation 530 and the relevant descriptions thereof.

The transmission module 440 may be configured to transmit information and/or instructions to one or more components of the surgery system 100. For example, the transmission module may transmit an instruction to the surgical equipment 120 to perform the surgical operation on the subject along the second route in the second coordinate system. Details regarding the transmission of the instruction may be found elsewhere in the present disclosure (e.g., operation 540 and the relevant descriptions thereof).

It should be noted that the above description of the processing device 140 is merely provided for the purpose of illustration, and not intended to limit the scope of the present disclosure. For persons having ordinary skills in the art, various variations and modifications may be performed in the light of the present disclosure. However, those variations and modifications do not depart from the scope of the present disclosure. For example, one or more of the modules of the processing device 140 mentioned above may be omitted or integrated into a single module. As another example, the processing device 140 may include one or more additional modules, for example, a storage module for data storage.

FIG. 5 is a flowchart illustrating an exemplary process for planning a surgical route for a surgical equipment according to some embodiments of the present disclosure. In some embodiments, the process 500 may be executed by the surgery system 100. For example, the process 500 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 500 presented below are intended to be illustrative. In some embodiments, the process may be accomplished with one or more additional operations not described, and/or without one or more of the operations discussed. Additionally, the order in which the operations of the process 500 as illustrated in FIG. 5 and described below is not intended to be limiting.

As used herein, the surgical route may refer to a route that the surgical equipment plans to travel through during performing a surgical operation on a subject. As described in connection with FIG. 1, exemplary surgical equipment may include a biopsy needle, a puncture needle, an ablation probe, a bone bit, a bone grinding tool, a surgical robot assembled with an actuating mechanism. Exemplary surgical operations may include a puncture, a biopsy, an ablation, a grinding, a drilling, an implantation, a suction. In some embodiments, the surgical route may pass through a plurality of physical points within or on the subject. The surgical route may be represented as (or correspond to) a set of coordinates of the physical points in one or more coordinate systems (e.g., the coordinate systems C0, C1 and C2 as shown in FIG. 1) or a vector in the one or more coordinate systems.

In 510, the processing device 140 (e.g., the obtaining module 410) (e.g., the interface circuits of the processor 220) may obtain a first image of the subject. The first image may be generated based on first scan data acquired by a first imaging device in a first coordinate system.

The subject may be a user, a portion of the user (e.g., an organ and/or a tissue of the user), a man-made object (e.g., a phantom), or the like, or any combination thereof. The first imaging device may be an imaging device 110, such as a CT device, a MRI device, a PET device, an X-ray imaging device, or the like. The first image may be a CT image, a MR image, a PET image, an X-ray image, or the like. The first image may be a 2-dimensional image, a 3-dimensional image, or a 4-dimensional image. In some embodiments, the first image may be a 3-dimensional CT image.

In some embodiments, the imaging device 110 may be operated to perform a first scan on the subject to generate the first scan data of the subject. The first image may be reconstructed based on the first scan data by, for example, the processing device 140. Alternatively, the first image may be previously generated based on the first scan data and stored in a storage device of the surgery system 100 (e.g., the storage device 150, the ROM 230, the RAM 240, or the storage 390). The processing device 140 may access the storage device and retrieve the first image of the subject. Alternatively, the first image of the subject may be obtained by the processing device 140 from an external source (e.g., a medical database) via the network 160.

In some embodiments, the first imaging device may correspond to the first coordinate system (e.g., the coordinate system C1) as described in connection with FIG. 1. The first image generated by the first imaging device may also correspond to the first coordinate system. The first image may include a plurality of voxels (or pixels) each of which has a coordinate in the first coordinate system. As used herein, a coordinate of the voxel (or pixel) of the first image in the first coordinate system may refer to a coordinate of a physical point of the subject corresponding to the voxel (or pixel) in the first coordinate system.

In some embodiments, the processing device 140 may determine the coordinates of the voxels (or pixels) in the first image in the first coordinate system based at least in part on the first image. For example, the subject may be placed in a predetermined position on a table of the first imaging device, wherein the predetermined position has a known coordinate in the first coordinate system and corresponds to a first voxel (or pixel) in the first image. The coordinate of a second voxel (or pixel) of the first image in the first coordinate system may be determined based on a relative position of the second voxel (or pixel) with respect to the first voxel (or pixel) in the first image. As another example, the tracking device 180 may acquire an image indicating a position of the subject in the first imaging device. The processing device 140 may determine the coordinates of the voxels (or pixels) of the first image based on the image and the first image. As yet another example, one or more markers may be deposited on a body surface of the first subject. The position(s) of the marker(s) in the first coordinate system (which may be denoted as coordinates of the marker(s) in the first coordinate system) may be tracked by the tracking device 180. The processing device 140 may determine the coordinates of the voxels (or pixels) of the first image based on the position(s) of the marker(s) in the first image and the coordinate(s) of the marker(s) in the first coordinate system. Details regarding the tracking device 180 may be found elsewhere in the present disclosure (e.g., FIG. 12 and the relevant descriptions thereof).

In some embodiments, the subject to may be moved to a certain position in a detection tunnel of the first imaging device to be scanned. The subject may remain at the certain position to receive the surgical operation. In this situation, the processing device 140 may determine the coordinates of the voxels (or pixels) in the first image in the first coordinate system when the subject is at the certain position. Alternatively, the subject may be moved to another position (e.g., a position outside the detection tunnel) to receive the surgical operation. In this situation, the processing device 140 may determine the coordinates of the voxels (or pixels) in the first image in the first coordinate system when the subject is at the another position.

In 520, the processing device 140 (e.g., the determination module 420) (e.g., the processing circuits of the processor 220) may determine a first route in the first image. The first route may extend from a first point of the subject to a second point of the subject in the first coordinate system.

As used herein, the first route may refer a virtual planned surgical route in the first image in the first coordinate system that corresponds to the surgical route of the surgical equipment. The first point and the second point in the subject may refer to two points of the subject in the first image that correspond to a first physical point and a second physical point within or on the subject, respectively. The first physical point may be a start point of the surgical route and the second physical point may be an end point of the surgical route. In some embodiments, the surgical equipment may be a puncture needle. The start point may also be referred to as a puncture point at which the puncture needle plans to puncture into the subject.

In some embodiments, the first point may be any point in the subject in the first image. For example, the first point may be a point on the body surface of the subject or a point within the subject. The second point may be any point within the subject in the first image. In some embodiments, the first point may be a point on the body surface of the subject in the first image and the second point may be a point in a lesion of the subject in the first image. Accordingly, the first route may correspond to a surgical route that penetrates the body surface of the subject to reach the lesion of the subject.

In some embodiments, the first route may be a linear or non-linear route. For example, if the surgical equipment may be a rigid equipment (e.g., a puncture needle), the first route may be linear route. If the surgical equipment may be a flexible equipment (e.g., a pipe), the first route may be non-linear route.

In some embodiments, the first route may pass through the first point, the second point, and one or more other points of the subject in the first image. The first route may be represented as a set of coordinates of the first point, the second point, and the other point(s) in the first coordinate system. Additionally or alternatively, the first route may be represented as a vector from the first point to the second point in the first coordinate system. In some embodiments, the processing device 140 may also determine one or more parameters associated with the first route in 520, such as a length of the first route, a direction of the first route (e.g., a direction represented as an angle between the first route and the X1/Z1 plane defined by the C1 coordinate system), a depth of the first route (e.g., a depth of the first route along the Y1 axis of the C1 coordinate system), or the like, or any combination thereof.

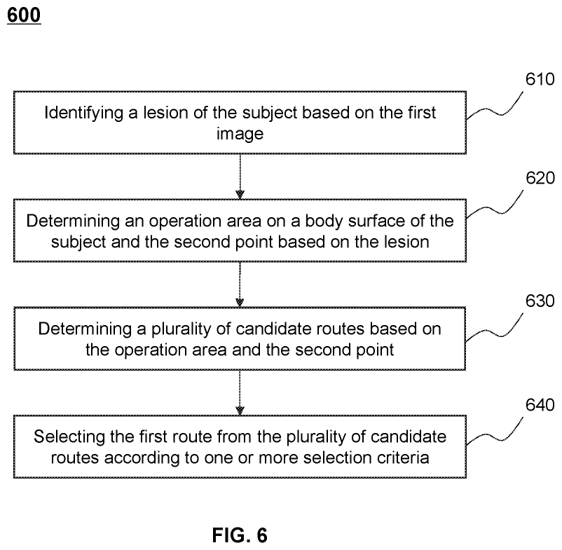

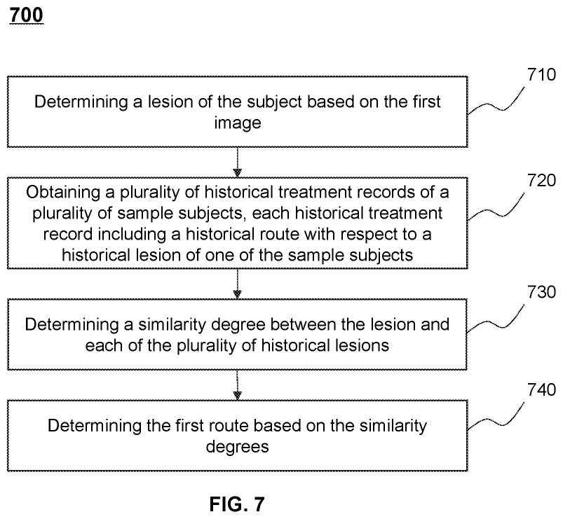

In some embodiments, the processing device 140 may determine the first route by one or more methods for determining the first route as disclosed in the present disclosure. For example, the first route may extend from a first point on the body surface to a second point at a lesion of the subject. The processing device 140 may determine an operation area on the body surface of the subject and the lesion of the subject based on the first image. The processing device 140 may further determine the first route based on the operation area and the lesion of the subject. As another example, the processing device 140 may determine the lesion of the subject based on the first image, and further determine the first route based on the lesion and a plurality of historical treatment records. Details regarding the determination of the first route may be found elsewhere in the present disclosure (e.g., FIGS. 6 and 7 and the relevant descriptions thereof).

In some embodiments, the processing device 140 may determine the first route under a user intervention. Merely by way of example, the processing device 140 may receive one or more parameters related to the first route from a user (e.g., a physician, a doctor). Exemplary parameters related to the first route may include a start point of the first route (also be referred to as the first point of the first route), an end point of the first route (also be referred to as the second point of the first route), a length of the first route, a direction of the first route, a depth of the first route, or the like. The processing device 140 may determine the first route according to at least one of the operation parameter(s) or in combination with one or more methods for determine the first route as disclosed in the present disclosure. As another example, the processing device 140 may determine a plurality of candidate first routes, and one of the candidate first routes may be selected as the first route by the user.

In 530, the processing device 140 (e.g., the transformation module 430) (e.g., the processing circuits of the processor 220) may transform the first route in the first coordinate system to a second route in a second coordinate system related to maneuvering of the surgical equipment.

As used herein, the second route may refer the actual planned surgical route of the surgical equipment in the second coordinate system. The surgical equipment may be maneuvered along the second route during the surgical operation. The second route may extend from the first physical point corresponding to the first point of the first route to the second physical point corresponding to the second point of the first route. In some embodiments, the second route may pass through the first physical point, the second physical point, and one or more other physical points of the subject. The second route may be represented as a set of coordinates of the first physical point, the second physical point, and the other physical point(s) in the second coordinate system. Additionally or alternatively, the second route may be represented as a vector from the first physical point to the second physical point in the second coordinate system. In some embodiments, the processing device 140 may also determine one or more parameters associated with the second route in 530, such as a length of the second route, a direction of the second route (e.g., a direction represented as a puncture angle between the second route and the body surface of the subject or the X2/Z2 plane defined by the C2 coordinate system as shown in FIG. 1), a depth of the second route (e.g., a depth of the second route along the Y2 axis of the C2 coordinate system), or the like, or any combination thereof. In some embodiments, the surgical operation may be a puncture operation. The direction of the second route may also be referred to as a puncture direction or a puncture angle.

In some embodiments, the processing device 140 may transform the first route to the second route based on a transformation relationship between the first coordinate system and the second coordinate system (also be referred to as a third transformation relationship). The transformation relationship between the first coordinate system and the second coordinate system may refer to a relationship between first coordinates of one or more points in the first coordinate system and their corresponding second coordinates in the second coordinate system. Take a specific point as an example, the transformation relationship may indicate a transformation relationship between a first coordinate of the specific point in the first coordinate system and a second coordinate of the specific point in the second coordinate system. The processing device 140 may determine the second coordinate of the specific point based on the first coordinate of the specific point and the transformation relationship between the first coordinate and the second coordinate.

In some embodiments, the transformation relationship the first coordinate system and the second coordinate system may be denoted in the form of a table recording the first coordinates of the one or more points in the first coordinate system and their corresponding second coordinates in the second coordinate system. Alternatively, the transformation relationship between the first coordinate system and the second coordinate system may be denoted in a transformation matrix or a transformation function.

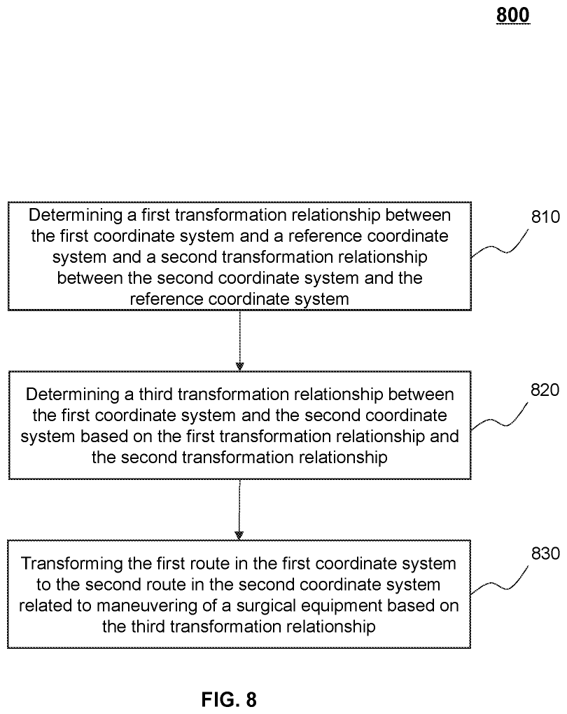

In some embodiments, the transformation relationship between the first coordinate system and the second coordinate system by be determined by the processing device 140 by performing one or more operations of process 800 as described in connection with FIG. 8. Alternatively, the transformation relationship between the first coordinate system and the second coordinate system may be previously determined by the processing device 140 or another computing device and stored in a storage device of the surgery system 100 (e.g., the storage device 150, the ROM 230, the RAM 240, or the storage 390). The processing device 140 may access the storage device and acquire the transformation relationship.

In 540, the processing device 140 (e.g., the transmission module 440) (e.g., the interface circuits of the processor 220) may transmit an instruction to the surgical equipment to perform the surgical operation on the subject along the second route in the second coordinate system.

As described in connection with FIG. 1, the surgical equipment may be an actuating mechanism. The instruction may direct the actuating mechanism to perform the surgical operation along the second route. Alternatively, the surgical equipment may be an equipment that assembled with the actuating mechanism. The instruction may direct the equipment to perform the surgical operation using the actuating mechanism (during the surgical operation, the actuating mechanism is directed to move along the second route). For example, the surgical equipment may be a surgical robot having a robotic arm, which assembled with an actuating mechanism (e.g., a puncture needle). The instruction may actuate the surgical robot to perform the surgical operation using the actuating mechanism. During the surgical operation, the actuating mechanism may be directed to move along the second route under the control of the robotic arm.

In some embodiments, the instruction may be transmitted to the surgical equipment via the network 160. The instruction may involve one or more parameters related to the second route, such as coordinates of the points in the second route in the second coordinate system, a direction of the second route, a length of the second route, a depth of the second route, or the like, or any combination thereof.

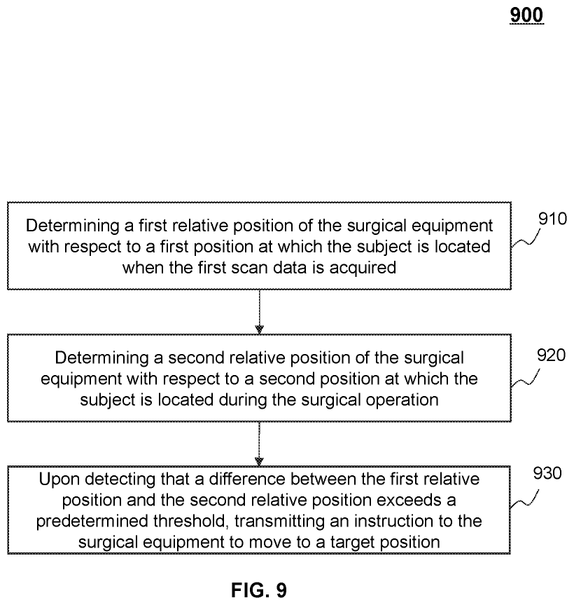

In some embodiments, after the first scan, the subject may remain at a position in the detection tunnel at which the subject undergoes the first scan. The surgical equipment or the actuating mechanism of the surgical equipment may reach into the detection tunnel to perform the surgical operation. Alternatively, the subject may be moved to a position outside the detection tunnel after the first scan. The surgical equipment or the actuating mechanism of the surgical equipment may perform the surgical operation outside the detection tunnel. In some embodiments, the processing device 140 may track a relative position between the surgical equipment and the subject during the first scan and the surgical operation to ensure that the surgical equipment remains at a stable relative position with respect the subject. Details regarding the tracking of the relative position may be found elsewhere in the present disclosure (e.g., FIGS. 9 and 12 and the relevant descriptions thereof).

In 550, the processing device 140 (e.g., the obtaining module 410) (e.g., the interface circuits of the processor 220) may obtain a second image of the subject after the surgical operation. The second image may be generated based on second scan data acquired by the first imaging device. The second image may be a 2-dimensional image, a 3-dimensional image, or the like, or any combination thereof. In some embodiments, the second image may be a 3-dimensional CT image.

In some embodiments, after the surgical operation is completed, the first imaging device may be operated to perform a second scan on the whole subject or a portion of the subject (e.g., a region of interest including the lesion of the subject) to generate the second scan data related to the subject. The second image may be reconstructed based on the second scan data. The obtaining of the second image may be performed in a similar manner with that of the first image as described in connection with 510, and the descriptions thereof are not repeated here.

In 560, the processing device 140 (e.g., the determination module 420) (e.g., the processing circuits of the processor 220) may determine an operation result based on the second image.

The operation result may include, for example, whether the lesion of the subject is removed by the surgical operation, whether a proportion of the lesion is removed by the surgical operation, whether the surgical equipment reaches the end point of the second route (i.e., the second physical point), or the like, or any combination thereof. In some embodiments, the processing device 140 may determine the operation result by comparing the first image (or the first scan data) with the second image (or the second scan data). For example, the processing device 140 may determine whether there is a lesion in the first image and whether there is a lesion in the second image. If there is a lesion in the first image but there is no lesion in the second image, the processing device 140 may determine that the lesion of the subject has been removed by the surgical operation. If there is a lesion in both the first and second images, the processing device 140 may further compare the sizes of the lesion in the two images to determine a proportion of the lesion that is removed. In some embodiments, the processing device 140 may transmit the second image to a terminal of a user, and the user may evaluate the operation result based on the second image.

In some embodiments, operations 540 to 560 may be performed for one or more iterations until a certain number of iterations are performed or the operation result in the current iteration satisfies a condition (e.g., the lesion is completely removed). In some embodiments, the subject may be placed at the same position (e.g., a specific position outside the detection tunnel) to receive the surgical operation in each iteration.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, the process 500 may include one or more additional operations or one or more of the operations mentioned above may be omitted. For example, any one of the operations 540 to 560 may be omitted. As another example, the process 500 may include one or more additional operations (e.g., one or more operations of process 900) to track the relative position between the subject and the surgical equipment. As yet another example, the process 500 may include one or more additional operations (e.g., one or more operations of process 1000) to monitor a moving trajectory of the surgical equipment during the surgical operation. In some embodiments, an operation of the process 500 may be divided into a plurality of sub-operations. For example, operation 530 may be divided into a first sub-operation in which the transformation relationship between the first coordinate system and the second coordinate system is determined and a second sub-operation in which the first route is transformed into the second route based on the transformation relationship.

FIG. 6 is a flowchart illustrating an exemplary process for determining a first route in a first image according to some embodiments of the present disclosure. In some embodiments, the process 600 may be executed by the surgery system 100. For example, the process 600 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 600 presented below are intended to be illustrative. In some embodiments, the process may be accomplished with one or more additional operations not described, and/or without one or more of the operations discussed. Additionally, the order in which the operations of the process 600 as illustrated in FIG. 6 and described below is not intended to be limiting. In some embodiments, one or more operations of the process 600 may be performed to achieve operation 520.

In 610, the processing device 140 (e.g., the determination module 420) (e.g., the processing circuits of the processor 220) may identify a lesion of the subject based on the first image.

A lesion may refer to an abnormal damage (or potential abnormal damage) or a change (or potential change) in a tissue or an organ of the subject. Exemplary lesions may include a tumor, an edema, a mass, or the like. In some embodiments, the processing device 140 may automatically identify the lesion and/or one or more other objects of interest (e.g., the skin surface and/or a soft tissue such as a vessel and a nerve) in the first image. The identification may be performed based on an imaging segmentation algorithm, such as a threshold-based segmentation algorithm, an edge-based segmentation algorithm, a region-based segmentation algorithm, a clustering-based algorithm, an image segmentation algorithm based on wavelet transform, an image segmentation algorithm based on mathematical morphology, an image segmentation algorithm based on artificial neural network, or the like, or any combination thereof. Optionally, the processing device 140 may mark the lesion on the first image and transmit the marked first image to a terminal (e.g., the terminal 130) of a user (e.g., a doctor, a physician) for display. The user may confirm or modify the lesion via the terminal. In some embodiments, the lesion may be identified manually by the user via the terminal. For example, the processing device 140 may transmit the first image to the terminal, and the user may mark the lesion on the first image via the terminal.

In some embodiments, the processing device 140 may further determine or obtain one or more features related to the lesion. Exemplary features related to the lesion may include the type of the lesion, the position of the lesion in the subject (e.g., represented by coordinates of one or more points of the lesion in the first coordinate system), the shape of the lesion, the size of the lesion, or the like, or any combination thereof.

In 620, the processing device 140 (e.g., the determination module 420) (e.g., the processing circuits of the processor 220) may determine an operation area on a body surface of the subject and the second point of the first route (i.e., the end point of the first route) based on the lesion.

The operation area on the body surface of the subject may refer to an area on the skin surface of the subject for performing the surgical operation, in which the first point of the first route (e.g., the start point of the first route) is located. The operation area may be any area on the body surface of the subject. The operation area may have any suitable shape and include any number of points. For example, the operation area may be the whole body surface, the whole front body surface, or the whole back body surface of the subject. As another example, the operation area may be an area on the body surface that is close to the lesion, for example, an area whose distance to the lesion is smaller than a threshold.

In some embodiments, the processing device 140 may determine the operation area by taking the position of the surgical equipment and/or a user into consideration. For example, the processing device 140 may determine an operation area close to the user, for example, an area whose distance to the user is smaller than a threshold. Additionally or alternatively, if the surgical equipment needs to reach into the detection tunnel of the first imaging device to perform the surgical operation, the processing device 140 may determine an operation area that the surgical equipment can reach.

The second point may be any point within or on the lesion, which may be the end point of the first route. In some embodiments, the second point may be a target of the lesion. In some embodiments, the second point may be determined automatically by the processing device 140. For example, the processing device 140 may determine the second point (e.g., the target of the lesion) by analyzing information related to the lesion (e.g., the type, the position, the size of the lesion). As another example, the processing device 140 may determine the second point based on a big data analyzing technique, for example, by referring to historical lesion data or using a machine learning model. Additionally or alternatively, the second point may be determined based on an input of a user via a terminal (e.g., the terminal 130). For example, the user may mark the second point on the first image via the terminal.

In 630, the processing device 140 (e.g., the determination module 420) (e.g., the processing circuits of the processor 220) may determine a plurality of candidate routes based on the operation area and the second point. Each of the plurality of candidate routes may extend from a point within the operation area to the second point.

In some embodiments, the operation area may include one or more points on the body surface of the subject. The processing device 140 may determine a candidate route extend from each point within the operation area (or a portion of the operation area) to the second point. Alternatively, the processing device 140 may segment the operation area into a plurality of sub-operation areas. The sizes of the sub-operation areas may be the same or different, which may be default values or be adjusted according to actual requirements (e.g., the size of the surgical equipment). The processing device 140 may further determine a candidate route corresponding to each sub-operation area, wherein the candidate route extends from a center point of the each sub-operation area to the second point. In some embodiments, the candidate routes may include one or more linear routes and/or one or more non-linear routes.

In 640, the processing device 140 (e.g., the determination module 420) (e.g., the processing circuits of the processor 220) may select the first route from the plurality of candidate routes according to one or more selection criteria.

Exemplary selection criteria may be related to the lengths of the candidate routes, the directions of the candidate routes, whether the candidate routes pass through one or more critical tissues of the subject, or the like, or any combination thereof. For example, the selection criteria may include that one or more candidate routes having the shortest N (e.g., 1, 3, 5, 10%, 20%) lengths among the candidate routes are selected, that one or more candidate routes having lengths smaller than a threshold or within a certain length range are selected, or the like. As another example, the selection criteria may include that one or more candidate routes not passing through one or more critical tissues of the subject (e.g., an organ, a blood vessel, a nerve) are selected. In some embodiments, the selection criteria may be default settings of the surgery system 100 or be manually set by a user of the surgery system 100.

In some embodiments, only one candidate route (e.g., the shortest candidate route) may be selected and the selected candidate route may be designated as the first route. Alternatively, a plurality of candidate routes may be selected. The processing device 140 may transmit the selected candidate routes to a terminal (e.g., the terminal 130) of a user (e.g., a doctor, a physician) of the surgery system 100. The user may choose one of the selected candidate routes as the first route. Alternatively, the user may choose one of the selected candidate routes and further modify the chosen candidate route, wherein the modified candidate route may be designated as the first route.

It should be noted that the above description of the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, in operation 620, the processing device 140 may determine the second point (e.g., the target of the lesion), and further determine a specific point on the body surface of the subject that has the shortest distance to the second point among all points on the body surface. The route extending from the specific point to the second point may be designated as the first route. In some embodiments, operations 630 and 640 may be combined into a single operation in which the processing device 140 determines the one or more candidate routes satisfying the selection criteria (e.g., not passing through one or more critical tissues of the subject). Then the first route may be selected from the one or more candidate routes by the processing device 140 or by the user of the surgery system 100.

FIG. 7 is a flowchart illustrating another exemplary process for determining a first route in a first image according to some embodiments of the present disclosure. In some embodiments, the process 700 may be executed by the surgery system 100. For example, the process 700 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 700 presented below are intended to be illustrative. In some embodiments, the process may be accomplished with one or more additional operations not described, and/or without one or more of the operations discussed. Additionally, the order in which the operations of the process 700 as illustrated in FIG. 7 and described below is not intended to be limiting. In some embodiments, one or more operations of the process 700 may be performed to achieve operation 520.

In 710, the processing device 140 (e.g., the determination module 420) (e.g., the processing circuits of the processor 220) may determine a lesion of the subject based on the first image. Operation 710 may be performed in a similar manner with operation 610, and the descriptions thereof are not repeated here.

In 720, the processing device 140 (e.g., the determination module 420) (e.g., the processing circuits of the processor 220) may obtain a plurality of historical treatment records related to a plurality of sample subjects. Each of the plurality of historical treatment records may include a historical route with respect to a historical lesion of a sample subject. Optionally, each historical treatment record may further include a historical image (e.g., a CT image or an X-ray image) of the sample subject and/or other information related to the historical lesion (e.g., the type, the position, the shape, and/or the size of the historical lesion). The historical route with respect to a historical lesion may be similar to the first route with respect to the lesion as described elsewhere in this disclosure (e.g., operation 520 and the relevant descriptions). The plurality of sample subjects may be of the same type of subject as the subject to be treated.

In some embodiments, the processing device 140 may obtain the historical treatment records according to one or more features of the lesion of the subject. For example, the historical lesions of the obtained historical treatment records may be of the same type or a similar type as the lesion of the subject. Additionally or alternatively, the positions of the historical lesions in the corresponding sample subjects may be similar to the position of the lesion in the subject, for example, both located at a same organ. In some embodiments, the historical treatment records or a portion thereof may be obtained from an external source (e.g., a medical database) via the network 160. Additionally or alternatively, the historical treatment records or a portion thereof may be obtained from a storage device of the surgery system 100, for example, the storage device 150, the ROM 120, and/or the RAM 240. The historical treatment records stored in the storage device may be historical treatment data of treatment subjects of the surgery system 100 and/or other medical systems. Optionally, the historical treatment records stored in the storage device may be processed by the processing device 140 or another computing device based on a machine learning technique. For example, one or more features (e.g., the type, the position, the shape, and/or the size) of the historical lesions may be extracted based on the machine learning technique.

In 730, the processing device 140 (e.g., the determination module 420) (e.g., the processing circuits of the processor 220) may determine a similarity degree between the lesion and each of the plurality of historical lesions.

In some embodiments, the processing device 140 may determine the similarity degrees between the lesion and the historical lesions by comparing one or more features of the lesion and the historical lesions. For example, for a specific historical lesion of a sample subject, the processing device 140 may determine the corresponding similarity degree by comparing the types of the lesion and the historical lesion and/or by comparing the position of the lesion in the subject and the position of the specific historical lesion in the sample subject. The processing device 140 may assign a higher similarity degree if the lesion and the specific historical lesion are of the same or similar type of lesion and/or located at similar positions. In some embodiments, the processing device 140 may compare the position of the lesion and the specific historical lesion by determining similarity points between the lesion and the specific historical lesion, the more similarity points are, the more similar the positions will be. As used herein, a first point of the lesion and a second point of the specific historical lesion may be regarded as similarity points if the position of the first point in the subject and the position of the second position in the sample subject is same or substantially same. For example, the processing device 140 may register the first image of the subject with a historical image of the sample subject and determine the similarity points of the lesion and the specific historical lesion based on the registration.

In some embodiments, the processing device 140 may determine a feature vector of the lesion and each of the historical lesions. The processing device 140 may further determine the similarity degree between the lesion and each of the historical lesions based on the feature vectors. For example, for a specific historical lesion of a sample subject, the processing device 140 may determine the corresponding similarity degree based on the feature vectors of the lesion and the specific lesion using a similarity algorithm. Exemplary similarity algorithms may include but be not limited to a Euclidean distance algorithm, a Manhattan distance algorithm, a Minkowski distance, a cosine similarity algorithm, a Jaccard similarity algorithm, a Pearson correlation algorithm, or the like, or any combination thereof. As another example, the processing device 140 may determine the corresponding similarity degree based on the feature vectors of the lesion and the specific lesion using a similarity model. The similarity model may be trained using historical data and used to determine a similarity degree between two lesions.

In 740, the processing device 140 (e.g., the determination module 420) (e.g., the processing circuits of the processor 220) may determine the first route based on the similarity degrees.

In some embodiments, the processing device 140 may selected one or more target route among the historical routes of the historical treatment records based on the similarity degrees. For example, the processing device 140 may select one or more historical lesions whose similarity degrees with the lesion are greater than a threshold, and designate the one or more historical routes corresponding to the selected historical lesions as the target route(s). Additionally or alternatively, the processing device 140 may rank the historical lesions according to the similarity degrees in, for example, a descending order. The processing device 140 may further select top N (e.g., 1, 3, 5, 10%, and 20%) historical lesion(s) among the historical lesions according to the ranking result, and designate the one or more historical routes corresponding to the selected historical lesions as the target route(s).

In some embodiments, only one historical route (e.g., the historical route whose corresponding historical lesion has the highest similarity degree with the lesion) may be selected, and the selected target route may be designated as the first route. In some embodiments, a plurality of target routes may be selected. The processing device 140 may further select the first route from the target routes. For example, the processing device 140 may select the first route from the target routes according to one or more selection criteria. The selection of the first route among the target routes may be performed in a similar manner with the selection of the first route among the candidate routes as described in connection with operation 640, and the descriptions thereof are not repeated here. As another example, the processing device 140 may transmit the target routes to a terminal (e.g., the terminal 130) of a user (e.g., a doctor, a physician) of the surgery system 100. The user may choose one of the target routes as the first route. Alternatively, the user may choose one of the target routes and further modify the chosen target route, wherein the modified target route may be designated as the first route.

It should be noted that the above description of the process 700 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 8 is a flowchart illustrating another exemplary process for transforming a first route in a first coordinate system to a second route in a second coordinate system according to some embodiments of the present disclosure. In some embodiments, the process 800 may be executed by the surgery system 100. For example, the process 800 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 800 presented below are intended to be illustrative. In some embodiments, the process may be accomplished with one or more additional operations not described, and/or without one or more of the operations discussed. Additionally, the order in which the operations of the process 800 as illustrated in FIG. 8 and described below is not intended to be limiting. In some embodiments, one or more operations of the process 800 may be performed to achieve operation 530.

In 810, the processing device 140 (e.g., the transformation module 430) (e.g., the processing circuits of the processor 220) may determine a first transformation relationship between the first coordinate system and a reference coordinate system and a second transformation relationship between the second coordinate system and the reference coordinate system.

As described elsewhere in this disclosure (e.g., FIGS. 1 and 5 and the relevant descriptions), the first imaging device, the surgical equipment, and the surgery system 100 may correspond to the first coordinate system, the second coordinate system, and the reference coordinate system, respectively. Similar to the third transformation relationship between the first coordinate system and the second coordinate system as described in connection with operation 540, the first transformation relationship between the first coordinate system and the reference coordinate system may refer to a relationship between first coordinates of one or more points in the first coordinate system and their corresponding reference coordinates in the reference coordinate system. The second transformation relationship between the second coordinate system and the reference coordinate system may refer to a relationship between second coordinates of one or more points in the second coordinate system and their corresponding reference coordinates in the reference coordinate system.

In some embodiments, the first transformation relationship and/or the second transformation relationship may be determined based on a plurality of markers placed on the body surface of the subject. The markers may include an optical marker, an RF marker, or a magnetic markers, or the like. For example, the processing device 140 and/or the tracking device 180 may determine a plurality of first coordinates, a plurality of second coordinates, and a plurality of reference coordinates of the markers in the first, the second, and the reference coordinate system, respectively. The processing device 140 may determine the first transformation relationship between the first coordinate system and the reference coordinate system based on the first coordinates and the reference coordinates. The processing device 140 may further determine the second transformation relationship between the second coordinate system and the reference coordinate system based on the second coordinates and the reference coordinates.

In some embodiments, the first coordinates may be denoted as a matrix T1, in which each element in the matrix T1 represents a first coordinate of a marker in the first coordinate system. The second coordinates may be denoted as a matrix T2, in which each element in the matrix T2 represents a second coordinate of a marker in the second coordinate system. The reference coordinates may be denoted as a matrix T3, in which each element in the matrix T3 represents a reference coordinate of a marker in the reference coordinate system. The first transformation relationship may be represented as a transformation matrix A between the matrixes T1 and T3. The second transformation relationship may be represented as a transformation matrix B between the matrixes T2 and T3. The transformation matrix A and/or the transformation matrix B may be determined according to a matrix transformation algorithm. In some embodiments, the first and/or the second transformation relationship may be represented as a first transformation function between the matrixes T1 and T3 and a second transformation function between the matrixes T2 and T3, respectively.

In 820, the processing device 140 (e.g., the transformation module 430) (e.g., the processing circuits of the processor 220) may determine a third transformation relationship between the first coordinate system and the second coordinate system based on the first transformation relationship and the second transformation relationship.

In some embodiments, the first and second transformation relationships may be represented as the transformation matrixes A and B, respectively. The third transformation relationship may be represented as a transformation matrix C between the transformation matrixes A and B. The processing device 140 may determine the transformation matrix C based on the transformation matrixes A and B according to a matrix transformation algorithm. In some embodiments, the third transformation relationship may be represented as a third transformation function between the transformation matrixes A and B.

In 830, the processing device 140 (e.g., the transformation module 430) (e.g., the processing circuits of the processor 220) may transform the first route in the first coordinate system to the second route in the second coordinate system based on the third transformation relationship.

In some embodiments, the first route may be represented as a set of coordinates of a plurality of points of the first route in the first coordinate system. The processing device 140 may transform the coordinate of each point of the first coordinate system to a corresponding coordinate of the point in the second coordinate system based on the third transformation relationship. Take a point M of the first route having a coordinate M1 in the first coordinate system as an example, the processing device 140 may transform the coordinate M1 to a corresponding coordinate M2 in the second coordinate system by, for example, multiplying M1 with the transformation matrix C or inputting M1 into the third transformation function. In some embodiments, the first route may be a vector in the first coordinate system. The processing device 140 may transform the vector in the first coordinate system to a corresponding vector in the second coordinate system by, for example, multiplying the vector with the transformation matrix C or inputting the vector into the third transformation function.

It should be noted that the above description of the process 800 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, operation 810 may be divided into a first sub-operation and a second sub-operation. In the first sub-operation, the processing device 140 may determine the first transformation relationship between the first and reference coordinate systems, for example, based on the first and reference coordinates of the markers placed on the subject. In the second sub-operation, the processing device 140 may determine the second transformation relationship between the second and reference coordinate systems, for example, based on the second and reference coordinates of the markers placed on the subject.

FIG. 9 is a flowchart illustrating another exemplary process for monitoring a relative position of a surgical equipment with respect to a subject according to some embodiments of the present disclosure. In some embodiments, the process 900 may be executed by the surgery system 100. For example, the process 900 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 900 presented below are intended to be illustrative. In some embodiments, the process may be accomplished with one or more additional operations not described, and/or without one or more of the operations discussed. Additionally, the order in which the operations of the process 900 as illustrated in FIG. 9 and described below is not intended to be limiting. In some embodiments, the process 900 may be jointly or separately performed by the tracking device 180 (or a processor thereof) and the processing device 140. For illustration purposes, the following descriptions are described with reference to the implementation of the process 900 by the processing device 140.

In some embodiments, the first scan may be performed on the subject when the subject is at an initial position in the surgery system 100. After the first scan is performed, the table of the first imaging device may be moved to a different position and the subject may be moved along with the table. Additionally or alternatively, the body of subject may be moved, for example, due to the respiratory of the subject. This may result in a change of the relative position between the surgical equipment and the subject. If the surgical route (i.e., the second route) of the surgical equipment is determined based on the first route in the first image and the transformation relationship between the first coordinate system and the second coordinate system, the surgical route may be unsuitable for the subject if the subject moves. Therefore, the relative position between the surgical equipment and the subject may need to be tracked to ensure that the surgical equipment remains at a stable relative position with respect to the subject during the first scan and the surgical operation.

In 910, the processing device 140 (e.g., the determination module 420) (e.g., the processing circuits of the processor 220) may determine a first relative position of the surgical equipment with respect to a first position at which the subject is located when the first scan data is acquired.

In 920, the processing device 140 (e.g., the determination module 420) (e.g., the processing circuits of the processor 220) may determine a second relative position of the surgical equipment with respect to a second position at which the subject is located during the surgical operation.

As used herein, the first position of the subject may refer to an initial position at which the subject undergoes the first scan. The first relative position may refer to a relative position between a third point on the surgical equipment and a fourth point on the body surface of the subject during the first scan. The second position at which the subject is located may refer to a current position of the subject during the surgical operation. The second relative position may refer to a relative position between the third point on the surgical equipment and the fourth point on the body surface of the subject during the surgical operation. The third point may be any point on the surgical equipment, for example, a point of a robotic arm of a surgical robot. The fourth point may be any point on the body surface of the subject, for example, a point within a predetermined distance to the lesion of the subject.

In some embodiments, the positions of the third and fourth points during the first scan may be represented as coordinates C3 and C4, respectively, in a specific coordinate system, such as the first coordinate system corresponding to the first imaging device, the second coordinate system corresponding to the surgical equipment, and/or the reference coordinate system corresponding to the surgery system 100. The first relative position may be represented as a first vector from C3 to C4 in the specific coordinate system. Similarly, the positions of the third and fourth points during the surgical operation may be represented as coordinates C3′ and C4′, respectively, in the specific coordinate system. The second relative position may be represented as a second vector from C3′ to C4′ in the specific coordinate system.

In some embodiments, the first and the second relative positions may be determined by tracking positions of one or more markers placed on the body surface of the subject and/or one or more markers placed on the surgical equipment. Details regarding the determination of the relative position between the surgical equipment and the subject may be found elsewhere in the present disclosure (e.g., FIG. 12 and the relevant descriptions thereof).

In 930, upon detecting that a difference between the first relative position and the second relative position exceeds a predetermined threshold, the processing device 140 (e.g., the determination module 420) (e.g., the processing circuits of the processor 220) may transmit an instruction to the surgical equipment to move to a target position. The relative position of the target position with respect to the second position of the subject may be substantially same as the first relative position with respect to the first position.

In some embodiments, the difference between the first relative position and the second relative position may refer to the difference between the first vector representing the first relative position and the second vector representing the second relative position. The difference between the first and second vectors may be measured by, for example, an angle between the first and second vectors, an Euclidean distance between the first and second vectors, a cosine similarity between the first and second vectors, or any parameter that can measure a difference or similarity between two vectors. In some embodiments, the predetermined threshold may be a default setting of the surgery system 100 or set manually by a user of the surgery system 100. In some embodiments, operation 930 may be performed simultaneously with operation 540.

It should be noted that the above description of the process 900 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, after the first scan is performed, the surgery system 100 (e.g., the processing device 140 and/or the tracking device 180) may determine the relative position between the surgical equipment and the subject continuously or periodically. If the change of the relative position exceeds the predetermined threshold, the surgical equipment may be instructed to move to a certain position to ensure that the surgical equipment locates at a stable relative position with respect to the subject.

In some embodiments, the movement of the subject after the first scan may be caused by the movement of the table of the first imaging device. For example, after the first scan is performed, the subject may be moved out from the detection tunnel of the first imaging device by the table of the first imaging device. After the surgical equipment is performed, the subject may be moved into the detection tunnel of the first imaging device again by the table for the second scan (as described in connection with operation 550). In some embodiments, the surgical equipment may be controlled to move consistently with the movement of the table and the subject so that the relative position between the surgical equipment and the subject may remain stable. For example, the processing device 140 may transmit instructions to the surgical equipment and the table, respectively, to direct the surgical equipment and the table to move in a consistent manner (e.g. move for a same distance in a same direction and same speed). As another example, before the second scan, the processing device 140 (e.g., the transmission module 440) (e.g., the processing circuits of the processor 220) may transmit an instruction to the first imaging device to move the subject into the detection tunnel via the table. During the subject being moved into the detection tunnel, the processing device 140 and/or the tracking device 180 may track a movement of the subject (or the table) periodically or continuously, for example, by tracking the one or more makers on the body surface of the subject. The movement of the subject may be defined by, for example, a movement distance, a movement speed, or the like, or any combination thereof. The processing device 140 and/or the tracking device 180 may transmit an instruction to the surgical equipment to move in a manner consistent with the movement of the subject. For example, the surgical equipment may be instructed to move a (substantially) same distance in a (substantially) same speed as the subject. In some embodiments, the surgical equipment may be an actuating mechanism assembled on a robotic arm of a surgical robot. The surgical robot may control the actuating mechanism to move consistently with the subject (or the table) via the robotic arm.

FIG. 10 is a flowchart illustrating another exemplary process for monitor a moving trajectory of a surgical equipment during a surgical operation according to some embodiments of the present disclosure. In some embodiments, the process 1000 may be executed by the surgery system 100. For example, the process 1000 may be implemented as a set of instructions (e.g., an application) stored in one or more storage devices (e.g., the storage device 150, the ROM 230, and/or RAM 240) and invoked and/or executed by the processing device 140 (implemented on, for example, the processor 220 of the computing device 200, the CPU 340 of the mobile device 300, and/or the modules illustrated in FIG. 4). The operations of the process 1000 presented below are intended to be illustrative. In some embodiments, the process may be accomplished with one or more additional operations not described, and/or without one or more of the operations discussed. Additionally, the order in which the operations of the process 1000 as illustrated in FIG. 10 and described below is not intended to be limiting. In some embodiments, the process 1000 may be performed periodically or continuously when the surgical equipment performs the surgical operation on the subject.

In 1010, the processing device 140 (e.g., the obtaining module 410) (e.g., the interface circuits of the processor 220) may obtain a third image of the subject. The third image may be generated according to scan data of the subject acquired by a second imaging device during the surgical operation. The third image may indicate a moving trajectory of the surgical equipment in the subject during the surgical operation.

The second imaging device may include any device that can capture the third image of the subject and the surgical equipment. In some embodiments, the second imaging device may be an ultrasonic imaging device or an X-ray imaging device. For example, the surgical equipment may be a surgical robot having one or more robotic arms, and the second imaging device may be an ultrasonic probe mounted on one of the robotic arms (e.g., an end of one of the robotic arms). As another example, the second imaging device may be a C-shaped X-ray imaging device placed at a certain position near the subject and the surgical equipment. In some embodiments, the second imaging device may be the first imaging device (e.g., a CT device or an MRI device) as described elsewhere in this disclosure (e.g., FIG. 5 and the relevant descriptions). The surgical operation may be performed when the subject are placed in the detection tunnel of the first imaging device, and the first imaging device may scan the subject during the surgical operation.

In some embodiments, the moving trajectory of the surgical equipment in the subject may be defined by one or more parameters of the surgical equipment. Exemplary parameters of the surgical equipment may include a position of the surgical equipment in the subject (e.g., a coordinate of the surgical operation in the second coordinate system), a movement direction of the surgical equipment, a depth of the surgical equipment in the subject, or the like, or any combination thereof. The processing device 140 may determine one or more of the parameters of the surgical equipment by analyzing the third image. In some embodiments, the second imaging device may be configured to capture an image of the subject during the surgical operation continuously or periodically. In this situation, the processing device 140 may obtain a plurality of third images of the subject. The processing device 140 may determine one or more of the parameters of the surgical equipment based on the plurality of third images, for example, determine the movement direction by comparing two consecutive third images.

In 1020, the processing device 140 (e.g., the determination module 420) (e.g., the processing circuits of the processor 220) may determine whether the moving trajectory of the surgical equipment deviates from the second route.

The second route may refer to the planned actual surgical route of the surgical equipment in the second coordinate system as described elsewhere in this disclosure (e.g., FIG. 5 and the relevant descriptions). In some embodiments, the processing device 140 may determine whether the moving trajectory of the surgical equipment deviates from the second route by comparing one or more parameters of the surgical equipment with the one or more parameters of the second route. Merely by way of example, the processing device 140 may determine whether the position of the surgical equipment indicated by the third image is in the second route or close to the second route (e.g., the distance between the position and the second route being smaller than a threshold). If the position of the surgical equipment is not in or close to the second route, the processing device 140 may determine that the moving trajectory of the surgical equipment deviates from the second route. Additionally or alternatively, the processing device 140 may determine whether the movement direction of the surgical equipment is parallel or substantially parallel with the direction of second route. If the direction of the surgical equipment is not parallel or substantially parallel with that of the second route, the processing device 140 may determine that the moving trajectory of the surgical equipment deviates from the second route. On the other hand, if the position of the surgical equipment is in or close to the second route and the direction of the surgical equipment is parallel or substantially parallel with that of the second route, the processing device 140 may determine that the moving trajectory of the surgical equipment is consistent with the second route.

In 1030, in response to a determination that the surgical equipment deviates from the second route, the processing device 140 (e.g., the transmission module 440) (e.g., the interface circuits of the processor 220) may transmit an instruction to the surgical equipment to terminate the surgical operation or adjust the surgical equipment.

In some embodiments, if the moving trajectory of the surgical equipment deviates from the second route, the surgical equipment may fail to accomplish an operation result and cause harm to the subject, for example, the surgical equipment may pass through one or more critical tissues near the lesion. This may be prevented by terminating or adjusting the surgical equipment after the processing device 140 detects that the deviation of the moving trajectory. In some embodiments, the processing device 140 may determine a degree of deviation of the moving trajectory with respect to the second route. The degree of deviation may be measured by, for example, a distance between the surgical equipment and the second route, a difference between the directions of the surgical equipment and the second route, or the like, or any combination thereof. If the degree of deviation exceeds a predetermined threshold, the processing device 140 may instruct the surgical equipment to terminate the surgical operation. If the degree of deviation does not exceed the predetermined threshold, the processing device 140 may instruct the surgical equipment to adjust the position and/or the movement direction of the surgical equipment.

In some embodiments, in response to a determination that the surgical equipment does not deviate from the second route, the surgical equipment may continue the surgical operation. The second imaging may continue to capture an image of the surgical equipment, and the moving trajectory of the surgical equipment may be monitored continuously until the surgical operation is finished.

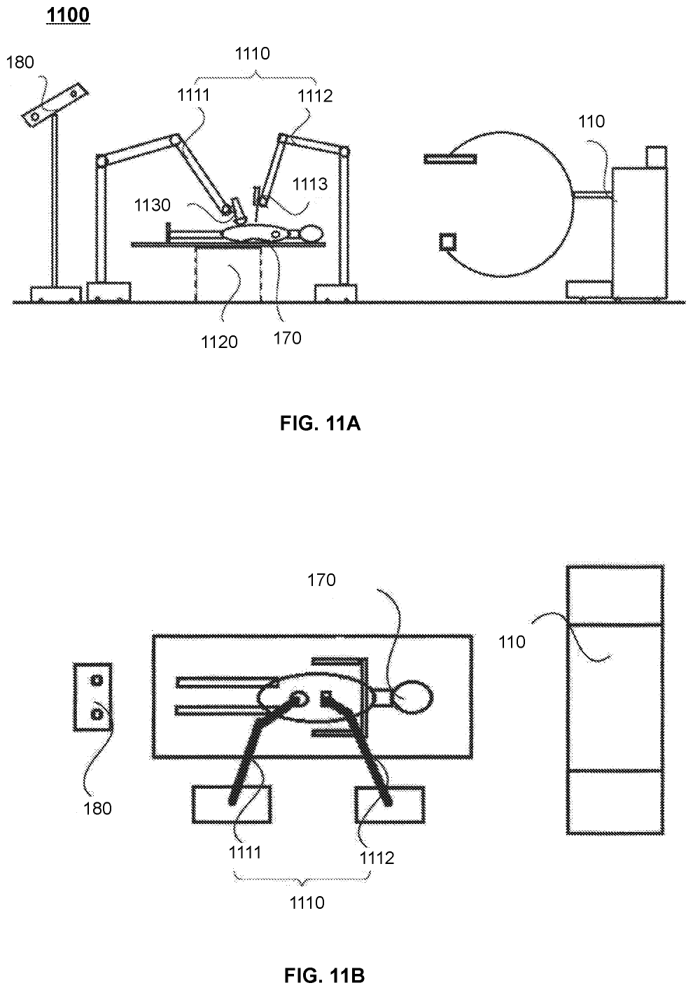

FIGS. 11A and 11B are schematic diagrams illustrating an exemplary surgical operation system according to some embodiments of the present disclosure. FIGS. 11A and 11B illustrate a front view and a top view of the surgery system surgery system 1100, respectively. In some embodiments, the surgery system 1100 may be an embodiment of the surgery system 100, which is configured to perform a surgical operation on the subject 170. As shown in FIGS. 11A and 11B, the surgical operation system 1100 may include an imaging device 110 (also referred to as the first imaging device), a table 1120, a tracking device 180, a surgical robot 1110, and an ultrasonic probe 1130 (also referred to as the second imaging device).

The imaging device 110 may be configured to perform a scan on the subject 170 to collect scan data related to the subject 170 before, during, and/or after the surgical operation. The surgical robot 1110 may be an embodiment of the surgical equipment 120 as shown in FIG. 1, which is configured to perform the surgical operation on the subject 170. The surgical robot 1110 may include a first robotic arm 1111, a second robotic arm 1112, and an actuating mechanism 1113 (e.g., a puncture needle) mounted on the second surgical robot 1112. The surgical robot 1110, the first robotic arm 1111, and the second robotic arm 1112 may be movable. In some embodiments, the surgical robot 1110 may further include a position detection device mounted on, for example, the actuating mechanism 1113 or the second robotic arm 1112. The position detection device may be configured to detect the position of the actuating mechanism 1113. For example, the position detection device may include a distance measuring device configured to measure a distance from the actuating mechanism 1113 to the subject 170 and/or an inclination angle measuring device configured to measure an inclination angle of the actuating mechanism 1113. In some embodiments, the position of the actuating mechanism 1113 may be transmitted to a processing device 140 (not shown in FIGS. 11A and 11B), and the processing device 140 may monitor the moving trajectory of the actuating mechanism 1113.

The table 1120 may be configured to support the subject. In some embodiments, the table 1120 may be movable and configured to move the subject to a desired position for a scan or the surgical operation. Optionally, the table 1120 may be integrated into the imaging device 110. The ultrasonic probe 1130 may be mounted on the first robotic arm 1111 configured to capture an image of the subject (e.g., the third image as described in connection with FIG. 10) during the surgical operation. The tracking device 180 may be configured to track positions of one or more components of the surgery system 1100. For example, the tracking device 180 may be a camera capturing an image or video of the surgery system 100, wherein the image or video may indicate the positions of the imaging device 110, the surgical robot 1110, and the subject 170 in the surgery system 1100.

It should be noted that the above description of the surgery system 1100 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the surgery system 1100 may include one or more additional components. For example, the surgery system 1100 may further include a processing device 140 configured to process data and/or information related to the surgery system 1100 and/or a terminal 130 configured to realize a user interaction with the surgery system 1100 (e.g., display the image captured by the ultrasonic probe 1130 in real-time). In some embodiments, one or more components of the surgery system 1100 described above may be omitted. For example, the first robotic arm 1111 may be omitted and the ultrasonic probe 1130 may be placed at any position at which the ultrasonic probe 1130 can capture the subject 170. As another example, the ultrasonic probe 1130 may be omitted and the imaging device 110 may be configured to scan the subject 170 during the surgical operation.

FIG. 12 is a schematic diagram illustrating an exemplary tracking device according to some embodiments of the present disclosure. As described elsewhere in this disclosure (e.g., FIGS. 1 and 9 and the relevant descriptions), the tracking device 180 may be configured to track the positions of one or more components of the surgery system 100 and/or determine relative positions between two or more components of the surgery system 100.

In some embodiments, the tracking device 180 may be an image acquisition device that captures an image or a video of the one or more components of the surgery system 100. For example, the tracking device 180 may be a camera (e.g., a binocular camera or a video camera), a mobile phone assembled with the camera, or the like, or any combination thereof. The tracking device 180 and/or a processing device (not shown in FIG. 12) may determine the positions of the one or more components and/or relative positions between two or more components based on the image or video. As another example, the tracking device 180 may determine the position of the one or more components by tracking one or more markers placed on the one or more components. The one or more markers may include an optical marker, an RF marker, a magnetic marker, or the like, or any combination thereof.

For illustration purposes, the tracking of the positions of the surgical equipment 120 and the subject 170 based on a plurality of optical markers is described as an example. As shown in FIG. 12, an optical marker 1210A is placed on the body surface of the subject 170 and an optical marker 1210B is placed on the surgical equipment 120. The optical marker 1210A may be placed at any position on the subject 170 and the optical marker 1210B may be placed at any position on the surgical equipment 120. For example, the optical marker 1210A may be placed on a region of interest (e.g., a lesion) of the subject and the optical marker 1210B may be placed on or close to the actuating mechanism of the surgical equipment 120.

In some embodiments, the optical markers 1210A and 1210B may include an optical source (e.g., an infrared source) that may emit light (e.g., infrared light). The tracking device 180 may receive the light emitted by the optical markers 1210A and 1210B. Alternatively, the optical markers 1210A and 1210B may be made of or coated with a reflective material. The tracking device 180 may include an optical source that may emit light toward the subject 170 and the surgical equipment 120, wherein the light may be reflected by the optical markers 1210A and 1210B and the reflected light may be received by the tracking device 180. The positions of the optical markers 1210A and 1210B (representing the positions of the subject 170 and the surgical equipment 120, respectively) may be determined by the tracking device 180 or the processing device 140 (not shown in FIG. 12) based on the light or reflected light received by the tracking device 180.

In some embodiments, the positions of the optical markers 1210A and 1210B may be denoted as coordinates of the optical markers 1210A and 12106 in one or more coordinate systems, such as the first, the second, and/or the reference coordinate system as described elsewhere in this disclosure. In some embodiments, the tracking device 180 or the processing device 140 may further determine a relative position between the surgical equipment 120 and the subject 170 based on the determined positions of the surgical equipment 120 and the subject 170. Details regarding the relative position between the surgical equipment 120 and the subject 170 may be found elsewhere in the present disclosure (e.g., FIG. 9 and the relevant descriptions thereof).

In some embodiments, a plurality of optical markers 1210A may be placed on the subject 170 and/or a plurality of optical marker 1210B may be placed on the surgical equipment 120. The positions of each optical marker 1210A or optical marker 1210B may be determined. The positions of the subject 170 and the surgical equipment 120 may be determined based on the positions of the optical markers 1210A and the positions of the optical markers 12106, respectively. For example, the position of the subject 170 may be represented as a position of a central point of the optical markers 1210A. The position of the surgical equipment 120 may be represented as a position of a central point of the optical markers 1210B. The relative position between the surgical equipment 120 and the subject 170 may be represented as the relative position between the two central points.

It should be noted that the above description of the surgery system 1200 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the optical marker 12106 may be omitted and the tracking device 180 may be mounted on the surgical equipment 120. The tracking device 180 may be configured to track the position of the optical marker 1210A placed on the subject 170. The position of the tracking device 180 may be regarded as the position of the surgical equipment 120. The relative position between the surgical equipment 120 and the subject 170 may be determined based on the position of the optical marker 1210A by the tracking device 180 or the processing device 140.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the descriptions, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
at least one storage medium including a set of instructions for surgical route planning; and
at least one processor configured to communicate with the at least one storage medium, wherein when executing the set of instructions, the at least one processor is configured to direct the system to perform operations including:
obtaining a first image of a subject, the first image being generated based on first scan data acquired by a first imaging device in a first coordinate system;
determining a first route in the first image, the first route extending from a first point of the subject to a second point of the subject in the first coordinate system;
transforming the first route in the first coordinate system to a second route in a second coordinate system related to maneuvering of a surgical equipment; and
transmitting an instruction to the surgical equipment to perform a surgical operation on the subject along the second route in the second coordinate system.

2. The system of claim 1, wherein to determine the first route in the first image, the at least one processor is further configured to direct the system to perform additional operations including:
identifying a lesion of the subject based on the first image;
determining an operation area on a body surface of the subject and the second point based on the lesion; and
determining the first route based on the operation area and the second point, wherein the first point is within the operation area.

3. The system of claim 2, wherein to determine the first route based on the operation area and the second point, the at least one processor is further configured to direct the system to perform additional operations including:
determining a plurality of candidate routes based on the operation area and the second point, each of the plurality of candidate routes extending from a point within the operation area to the second point; and
selecting the first route from the plurality of candidate routes.

4. The system of claim 3, wherein the selection of the first route is based on one or more selection criteria, and the one or more selection criteria are related to at least one of lengths of the plurality of candidate routes, directions of the plurality of candidate routes, or whether the plurality of candidate routes pass through one or more critical tissues of the subject.

5. The system of claim 1, wherein to determine the first route in the first image, the at least one processor is further configured to direct the system to perform additional operations including:
identifying a lesion of the subject based on the first image;
obtaining a plurality of historical treatment records of a plurality of sample subjects, each of the plurality of historical treatment records including a historical route with respect to a historical lesion of one of the plurality of sample subjects; and
determining the first route based on the lesion and the plurality of historical treatment records.

6. The system of claim 5, wherein to determine the first route based on the lesion and the plurality of historical records, the at least one processor is further configured to direct the system to perform additional operations including:
determining a similarity degree between the lesion and each of the plurality of historical lesions; and
determining the first route based on the similarity degrees.

7. The system of claim 1, wherein to determine the first route in the first image, the at least one processor is further configured to direct the system to perform additional operations including:
receiving one or more operation parameters related to the first route from a user; and
determining the first route based at least one of the one or more operation parameters.

8. The system of claim 1, wherein to transform the first route in the first coordinate system to the second route in the second coordinate system related to maneuvering of the surgical equipment, the at least one processor is further configured to direct the system to perform additional operations including:
determining a first transformation relationship between the first coordinate system and a reference coordinate system;
determining a second transformation relationship between the second coordinate system and the reference coordinate system;
determining a third transformation relationship between the first coordinate system and the second coordinate system based on the first transformation relationship and the second transformation relationship; and
transforming the first route in the first coordinate system to the second route in the second coordinate system related to maneuvering of a surgical equipment based on the third transformation relationship.

9. The system of claim 8, wherein to determine the first transformation relationship between the first coordinate system and the reference coordinate system, the at least one processor is further configured to direct the system to perform additional operations including:
determining a plurality of first coordinates of a plurality of markers placed on a body surface of the subject in the first coordinate system;
determining a plurality of reference coordinates of the plurality of markers in the reference coordinate system; and
determining the first transformation relationship between the first coordinate system and the reference coordinate system based on plurality of first coordinates and the plurality of reference coordinates.

10. The system of claim 9, wherein to determine the second transformation relationship between the second coordinate system and the reference coordinate system, the at least one processor is further configured to direct the system to perform additional operations including:
determining one or more second coordinates of the one or more markers in the second coordinate system; and
determining the second transformation relationship between the second coordinate system and the reference coordinate system based on the one or more second coordinates and the one or more reference coordinates.

11. The system of claim 1, wherein the at least one processor is further configured to direct the system to perform additional operations including:
determining a first relative position of the surgical equipment with respect to a first position at which the subject is located when the first scan data is acquired;
determining a second relative position of the surgical equipment with respect to a second position at which the subject is located during the surgical operation; and
upon detecting that a difference between the first relative position and the second relative position exceeds a predetermined threshold, transmitting an instruction to the surgical equipment to move to a target position, the target position having a substantially same relative position with respect to the second position of the subject as the first relative position with respect to the first position.

12. The system of claim 11, wherein:
at least one of the first relative position or the second relative position is determined by tracking positions of at least one of one or more first makers placed on a body surface of the subject or one or more second markers placed on the surgical equipment.

13. The system of claim 1, wherein the at least one processor is further configured to direct the system to perform additional operations including:
obtaining a second image of the subject after the surgical operation, the second image being generated based on second scan data acquired by the first imaging device; and
determining an operation result based on the second image.

14. The system of claim 13, wherein to obtain the second image of the subject after the surgical equipment, the at least one processor is further configured to direct the system to perform additional operations including:
transmitting an instruction to the first imaging device to move the subject into a detection tunnel of the first imaging device;
determining a movement of the subject during moving the subject into the detection tunnel; and
transmitting an instruction to the surgical equipment to move in a manner consistent with the movement of the subject.

15. The system of claim 1, wherein the at least one processor is further configured to direct the system to perform additional operations including:
obtaining a third image of the subject, the third image being generated according to scan data acquired by a second imaging device during the surgical operation, the third image indicating a moving trajectory of the surgical equipment during the surgical operation;
determining whether the moving trajectory of the surgical equipment deviates from the second route; and
in response to a determination that the surgical equipment deviates from the second route, transmitting an instruction to the surgical equipment to terminate the surgical operation or adjust the surgical operation.

16. The system of claim 15, wherein the surgical equipment is mounted on a first robotic arm of a surgical robot, and the second imaging device is an ultrasonic imaging device mounted on a second robotic arm of the surgical robot.

17. The system of claim 1, wherein the surgical operation includes at least one of a puncture, a biopsy, an ablation, a grinding, a drilling, an implantation, or a suction.

18. The system of claim 1, wherein the first imaging device is a computed tomography (CT) device or a multi-modality imaging device including the CT device.

19. A method, implemented on a computing device having one or more processors and one or more storage media, the method comprising:
obtaining a first image of a subject, the first image being generated based on first scan data acquired by a first imaging device in a first coordinate system;
determining a first route in the first image, the first route extending from a first point of the subject to a second point of the subject in the first coordinate system;
transforming the first route in the first coordinate system to a second route in a second coordinate system related to maneuvering of a surgical equipment; and
transmitting an instruction to the surgical equipment to perform a surgical operation on the subject along the second route in the second coordinate system.

20-36. (canceled)

37. A non-transitory computer readable medium, comprising a set of instructions for surgical route planning, wherein when executed by at least one processor, the set of instructions directs the at least one processor to:
obtain a first image of a subject, the first image being generated based on first scan data acquired by a first imaging device in a first coordinate system;
determine a first route in the first image, the first route extending from a first point of the subject to a second point of the subject in the first coordinate system;
transform the first route in the first coordinate system to a second route in a second coordinate system related to maneuvering of a surgical equipment; and
transmit an instruction to the surgical equipment to perform a surgical operation on the subject along the second route in the second coordinate system.

38. (canceled)
